SEQUENCE LISTING (62 sequences)

SEQ ID NO: 1 - DNA, 10953 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttggaggcttgctgaaggctgtatgctgttgtcggggccggtttcggggccttagtgaagccacagatgtacggccccgaaaccggccccaggacacaaggcctgttactagcactcacatggaacaaatggccaccgtgggaggatgacaagtgatatcacaaggtcccagggctggggtcagaaattctctcccgagggaatgaagccacaggagccaagagcaggaggaccaaggccctggcgaaggccgtggcctcgttcaagtaaaagatcctagtacagtgcaggtcccaatgtgtactaggatcttttacttgaacggggacgccggcatccgggctcaggacccccctctctgccagaggcaccaacaccagagttcacaaatcagtctcctgccctttgcatgtagcaaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaatgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 2 - DNA, 10848 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttggaggcttgctgaaggctgtatgctgttgtcggggccggtttcggggccttagtgaagccacagatgtacggccccgaaaccggccccaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 3 - DNA, 1446 bp, Artificial Sequence
  Synthetic Polynucleotide
atgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaa

SEQ ID NO: 4 - protein, 481 aa, Artificial Sequence
  Synthetic Polypeptide
MSTLCPPPSPAVAKTEIALSGKSPLLAATFAYWDNILGPRVRHIWAPKTEQVLLSDGEITFLANHTLNGEILRNAESGAIDVKFFVLSEKGVIIVSLIFDGNWNGDRSTYGLSIILPQTELSFYLPLHRVCVDRLTHIIRKGRIWMHKERQENVQKIILEGTERMEDQGQSIIPMLTGEVIPVMELLSSMKSHSVPEEIDIADTVLNDDDIGDSCHEGFLLNAISSHLQTCGCSVVVGSSAEKVNKIVRTLCLFLTPAERKCSRLCEAESSFKYESGLFVQGLLKDSTGSFVLPFRQVMYAPYPTTHIDVDVNTVKQMPPCHEHIYNQRRYMRSELTAFWRATSEEDMAQDTIIYTDESFTPDLNIFQDVLHRDTLVKAFLDQVFQLKPGLSLRSTFLAQFLLVLHRKALTLIKYIEDDTQKGKKPFKSLRNLKIDLDLTAEGDLNIIMALAEKIKPGLHSFIFGRPFYTSVQERDVLMTF

SEQ ID NO: 5 - DNA, 10900 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtcttgtcatcctcccacggtggccatttgttccatgtgagtgctagtaacaggccttgtgtcctggggccggtttcggggccgtacatctgtggcttcactaaggccccgaaaccggccccgacaacagcatacagccttcagcaagcctccagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaatgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 6 - protein, 274 aa, Artificial Sequence
  Synthetic Polypeptide
MGKSLSHLPLHSSKEDAYDGVTSENMRNGLVNSEVHNEDGRNGDVSQFPYVEFTGRDSVTCPTCQGTGRIPRGQENQLVALIPYSDQRLRPRRTKLYVMASVFVCLLLSGLAVFFLFPRSIDVKYIGVKSAYVSYDVQKRTIYLNITNTLNITNNNYYSVEVENITAQVQFSKTVIGKARLNNITIIGPLDMKQIDYTVPTVIAEEMSYMYDFCTLISIKVHNIVLMMQVTVTTTYFGHSEQISQERYQYVDCGRNTTYQLGQSEYLNVLQPQQ

SEQ ID NO: 7 - DNA, 6514 bp, Artificial Sequence
  Synthetic Polynucleotide
aggcgcggacgcaggttacagcagcgcttggcctctgctgatgccgtcgttatcctacccctcccccgtcccagctctacggcggccgcgcgctccaggccggtcgctccaccccccggctcccgggactgtggactccacgaccctgtcctcggccctgtccgcgccgaagcagcccgggactgcgcagcgccccgcgtgccgatcttttcctaattcagcagcgatttaaccaagagcctggaatattttaaggagtaataagagacatttacaaactattctctctgaagcctgctacctggaggcatcatctagataatcagaaccttggcttccacatcctcctcccttgtcttaactacaaacatttctttctgctgacttcaactcctcagacatgggaaagtctctttctcatttgcctttgcattcaagcaaagaagatgcttatgatggagtcacatctgaaaacatgaggaatggactggttaatagtgaagtccataatgaagatggaagaaatggagatgtctctcagtttccatatgtggaatttacaggaagagatagtgtcacctgccctacttgtcagggaacaggaagaattcctagggggcaagaaaaccaactggtggcattgattccatatagtgatcagagattaaggccaagaagaacaaagctgtatgtgatggcttctgtgtttgtctgtctactcctttctggattggctgtgtttttccttttccctcgctctatcgacgtgaaatacattggtgtaaaatcagcctatgtcagttatgatgttcagaagcgtacaatttatttaaatatcacaaacacactaaatataacaaacaataactattactctgtcgaagttgaaaacatcactgcccaagttcaattttcaaaaacagttattggaaaggcacgcttaaacaacataaccattattggtccacttgatatgaaacaaattgattacacagtacctaccgttatagcagaggaaatgagttatatgtatgatttctgtactctgatatccatcaaagtgcataacatagtactcatgatgcaagttactgtgacaacaacatactttggccactctgaacagatatcccaggagaggtatcagtatgtcgactgtggaagaaacacaacttatcagttggggcagtctgaatatttaaatgtacttcagccacaacagtaaaaactggaagagatggatttaaagaagaaatatctattgatatttcctatactctcaatgaagaggtatttcctaataggagaccttaaattgaacaaacctaaagtttacacttctaagagtacagttaaaagtatgtggacctgcagttcttgtaactctccactctgtgttaatgatatatttgtactaggatcttttacttgaatctaaatttactggttgatttccttctccagcctatcccctacagggaaaagctgatacttcccctatagtacaataaataattatttaaaagtcatagctccagtcactactgaaaacataattttggtgataaacataatttgagaaacttaatttctgaatgtttttatagaaaattactgaaagtctattactcatggaagacttttaaagaataaccttttttcctgttttataaattcccattgttatatggtagtatttcagctacacaatattttagcttttagctagacatttatagcttttcatttgttgaaatggtaatcatctgcatgtttttgtcacttatttcaggttagtgattgcctaacacttataagccaaaataatctttgcaaaattccatacctaaaattttgaaagcccctaatgttttcacacatctttctgtattagttatagttttgtgaaatctttgtgtgatcttcaaacattatcatttaatgtacaatactgtaaataaactgtgcatggcttttatacagctttagtaaatgtcaaataaagtggtacagactcattacaacaagtttctcataaaaatacaataaataggaaaatgaaattcagaaacccatagactgggaataggttccagttacagcttggatctggcataaaataaatttgaaataaaatattttgatgctccatttttttatgttgcttttcatactaaagaatggtgtagacatgttttgcaactgttaggtacccagttatcaattttatcaatgttttagaggaggaaattatttttttggtagaaattgttcaagaaatccttaattgaatgtcattaaatgatggtggccaaaataaaacctatttagaaatttaatcactttgcacatcacttggaatatgatgcctctagtagttacttttttatagttttctacttttggttttatttaaaattgttttcaaatatagattattgacttattcaactttgctgttttatattttcagtatcatttttcatttttttttttttttgtcttttcacttaccaagttctagggacatttaaaatatgtactaagtgtaggagtggttatgataccaaaaaatgtagctgggttgagattaatttcgttctgttttctcatgacagaaatcaggtttccctttccccacccctaagtgcctaacttaggtctgaaacagcctgtttattagtctgactctctcaaccataaaacataagctttatttaattctgcctttaaacacactcaggtttccccttaattttcatattattttctgcaagttttcttgagtatcttcaattcgttgaatgtggtttttggtttttttttgttttaacactagtcttcccttaattcattgctaactcaagccatccttactattaaacccaaatcagtcctttaagttcattatggcctttctagtatttaaaaaaaaaaaaatcattttcatttttcttctgctacgtttcctgactactactgcatacttctctgatacaggttctgtttgtattttttatatcattctcattttctcatttgacatgatctatgtctatatatgatataggtccccttttgtctcaaaatttttaattatgtgacttcaaaaatcacctgtatctgtagtagggcttccaaatctgcttctccatatgtgaccagtcacctgtctgctttcacatttagctagtgaactacacatttactaaaatgtgtaaattttacacatttagtgactgtgtaaaataaaaaaaaagttattttatcatatcctttctattatgttcccatcctgtcctcatgtcccatttactttattatcaccattcatttcttcaaaattatcttttagatacgctcatacaaaaatcaatccttgttttcttgcttgtgtcttttaaccttggaaaattacatcgtgtaaattaaacagatttttctgatgatctgtgcttcttatatactattagagtgcatgatagtatctcctgaaaaggatggaaagtagaagcatttgcttttagtcacttaattttgaatcttttttcttcatcttttgaattaattttttttattatatctacttttagtggagtttgagtcagaaaaaaacaagaatttgaaacaagtaaaaagatagaagagaaataaagatggtatgtgactactttcagagagagttaagtaactgtcagaataagcctggaacaaaacaggctgtaaattaataaaactacaaacacacattcaggtgaagcagaagtatagccataaaacatctagaaagagtgaatgaggcttttagcttttcttaggtcaatgtccagtgtgcttttttccatgggaataggataggtattaatacgcttttctaaactgctctcagaccttatccagaggacatggtaaagatatgttacagaaatttttctgatacttcctggaataactttaagttacaccctagtagactggtcattctaataaaatccagtactataacaaacctctgtatgttgatagcacattggccctttttagagttctttcctatgtttttcttacgtgatttcccacagttccatgagtccaacaaaggagagtgataggctccttatcttttagaagaggaaggaaaggcatgaagaagttgagggactggctgaagatcacgtacttactaagtagtacaactggagcaagatcaagtatctctgtctcccatatctgtgttctatcatttaaaatatatattggaaatccctgctgactcagattggtatgattaaaaatgagaggaaagttcaaatagttagtagtgacaaactaatactgctggactaagattttggtagcattgttttctaaaatattttaaatggagaatgaacacttataaaatgctttggaacataatctttagcttaattttctgttaaaatttagtaccccttcatcattccaataaagataagactgatccattgtctaaggaaattatttataaataatagagattaatttatttgagatttgaaataagaatagtatgaaaatattagataccacataaattgtttgaaattactgaataaccatcttaagtatggaacatttaaatggctatattttatttgtgtacagtttttctgtgccttgttaggccagtgaagcaattattttctctaagaaaatgacaataaaatataacacacttcagattgtctgatttacagtttggaaaggacaccgcaatgttcaaataggtaggagaccatcaaaaacacaattaaagtaacatattaggagacttgaaacttcagcctaataaatccttcatggttcttagccttattattgtgatataattctagatattttcttggagggcatgtgcccaactctcccgcaccccattttgtttgtcttttaaagttcttagaataaacagttctttatataataattatattttatttaagaaaatagtttgttaggtactttttaaaagatgtaaatttttaaatttacaaatacatatgggtctttgataagcaataggaattgaattacaagttactagggttataagcaaaaggttgcttaccataatgtcattaggtcacgatttttagctcacatctggaagcagcaactacttggctcaagtacatataagagtaattagttttattctctcttttttataaaatcgggtttcagatgagatgtttatcttagactattttagggaaaaattttacatgtttgagatggtggagtaaaaagactgttaaacatttcttttaaaaaattatttttacattacaacaatatatttatgatgtgttcagatcaaaaatttaacttctgtgtcccagatctactttcaaagtgagattttcacttgtcagcttaaatttctgactagaactaacatttgtgtatttttgtgcttagtcggaatacaaatttcacagtggatttttgaagtttgtccttaaattggataaaatcaagtgattaaagttactaaagagataaaaatggtaatttccatttttaaaagtaatttggttgtgtttatagttatttgtacaagtatttatcacagactctaaattgaaaaatgtagtatgatctatatttgaccctaaaaatgttggattaatttaacaaatatggcagatttttcataactaagtcttaagtcttctaaaaggaagctgttacccttctgtttttaattacattaattgaaatgtgttttaagagatacaatttcagcatattttatatattaaaaaacaaaaaaggattagtattgagccagtggccaaaaggtaatattactaccatgtagactgttatagttcaaattgtcccacttcacccagaattttagaaactagaagtctgggaggtactatatcagctgtagttgggtaattccaagtgctgatagtactattcatcttttttattattgtgtcagatgaaacaaatgccaagttgcaaaatatgcagatttttattatataatggttttaggcataaattattaacaagccatgccttatgtgtttcatcttatatttttctttagaactaaactataacagattttggaaaatgatttgacgtgcttgctcacttgattgacttggtcagatatttgaatgatggtattacctagattctaatccttgattctagttatataataaataatatagaatatgaaaatatgtttgggcatttactgtttatattatgtagtagcctccatcatgacacacttactacatttatgaattgagcagttctgtaattgtaattattattgctgttcatgtaacaaaacatgcttataatagcaaacaaatagaaatgcccccaaaatgctatttttttaattcagttataactgttactcttgtagttgtgtatgacgcaataaaatttgtaaaaaaatttcagcatgaaaaataaaatttgtatcacttatgta

SEQ ID NO: 8 - protein, 1313 aa, Artificial Sequence
  Synthetic Polypeptide
MRSAAAAPRSPAVATESRRFAAARWPGWRSLQRPARRSGRGGGGAAPGPYPSAAPPPPGPGPPPSRQSSPPSASDCFGSNGNGGGAFRPGSRRLLGLGGPPRPFVVLLLPLASPGAPPAAPTRASPLGARASPPRSGVSLARPAPGCPRPACEPVYGPLTMSLKPQQQQQQQQQQQQQQQQQQQQQQQPPPAAANVRKPGGSGLLASPAAAPSPSSSSVSSSSATAPSSVVAATSGGGRPGLGRGRNSNKGLPQSTISFDGIYANMRMVHILTSVVGSKCEVQVKNGGIYEGVFKTYSPKCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYACPKLPYNKETSPSFYFAISTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQAHVQSGMVPSHPTAHAPMMLMTTQPPGGPQAALAQSALQPIPVSTTAHFPYMTHPSVQAHHQQQL

SEQ ID NO: 9 - DNA, 4712 bp, Artificial Sequence
  Synthetic Polynucleotide
acccccgagaaagcaacccagcgcgccgcccgctcctcacgtgtccctcccggccccggggccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 10 - DNA, 19 bp, Artificial Sequence
  Synthetic Polynucleotide
ttggcctttcggttcctcc

SEQ ID NO: 11 - DNA, 19 bp, Artificial Sequence
  Synthetic Polynucleotide
ggaggaaccgaaaggccaa

SEQ ID NO: 12 - DNA, 148 bp, Artificial Sequence
  Synthetic Polynucleotide
tggaggcttgctgaaggctgtatgctgttgtcttggcctttcggttcctccagtgaagccacagatgggaggaaccgaaaggccaaaggacacaaggcctgttactagcactcacatggaacaaatggccaccgtgggaggatgacaa

SEQ ID NO: 13 - DNA, 148 bp, Artificial Sequence
  Synthetic Polynucleotide
ttgtcatcctcccacggtggccatttgttccatgtgagtgctagtaacaggccttgtgtcctttggcctttcggttcctcccatctgtggcttcactggaggaaccgaaaggccaagacaacagcatacagccttcagcaagcctcca

SEQ ID NO: 14 - DNA, 22 bp, Artificial Sequence
  Synthetic Polynucleotide
atagcagcaagaatcactcttg

SEQ ID NO: 15 - DNA, 22 bp, Artificial Sequence
  Synthetic Polynucleotide
caagagtgattcttgctgctat

SEQ ID NO: 16 - DNA, 151 bp, Artificial Sequence
  Synthetic Polynucleotide
ctggaggcttgctttgggctgtatgctgatagcagcaagaatcactcttgttttggcctctgactgaacaagaggatcttgctgctatcaggacacaaggccctttatcagcactcacatggaacaaatggccaccgtgggaggatgacaa

SEQ ID NO: 17 - DNA, 151 bp, Artificial Sequence
  Synthetic Polynucleotide
ttgtcatcctcccacggtggccatttgttccatgtgagtgctgataaagggccttgtgtcctgatagcagcaagatcctcttgttcagtcagaggccaaaacaagagtgattcttgctgctatcagcatacagcccaaagcaagcctccag

SEQ ID NO: 18 - DNA, 300 bp, Artificial Sequence
  Synthetic Polynucleotide
tggaggcttgctgaaggctgtatgctgttgtctactgctctatatgtcttggcagtgaagccacagatggccaagacatatagagcagtaaggacacaaggcctgttactagcactcacatggaacaaatggccaccgtgggaggatgacaatggaggcttgctgaaggctgtatgctgttgtcttggcctttcggttcctccagtgaagccacagatgggaggaaccgaaaggccaaaggacacaaggcctgttactagcactcacatggaacaaatggccaccgtgggaggatgacaa

SEQ ID NO: 19 - DNA, 300 bp, Artificial Sequence
  Synthetic Polynucleotide
ttgtcatcctcccacggtggccatttgttccatgtgagtgctagtaacaggccttgtgtcctttggcctttcggttcctcccatctgtggcttcactggaggaaccgaaaggccaagacaacagcatacagccttcagcaagcctccattgtcatcctcccacggtggccatttgttccatgtgagtgctagtaacaggccttgtgtccttactgctctatatgtcttggccatctgtggcttcactgccaagacatatagagcagtagacaacagcatacagccttcagcaagcctcca

SEQ ID NO: 20 - DNA, 23 bp, Artificial Sequence
  Synthetic Polynucleotide
ttccagttggtagaagcagtaga

SEQ ID NO: 21 - DNA, 23 bp, Artificial Sequence
  Synthetic Polynucleotide
tctactgcttctaccaactggaa

SEQ ID NO: 22 - DNA, 152 bp, Artificial Sequence
  Synthetic Polynucleotide
ctggaggcttgctttgggctgtatgctgttccagttggtagaagcagtagattttggcctctgactgatctactgttcaccaactggaacaggacacaaggccctttatcagcactcacatggaacaaatggccaccgtgggaggatgacaa

SEQ ID NO: 23 - DNA, 152 bp, Artificial Sequence
  Synthetic Polynucleotide
ttgtcatcctcccacggtggccatttgttccatgtgagtgctgataaagggccttgtgtcctgttccagttggtgaacagtagatcagtcagaggccaaaatctactgcttctaccaactggaacagcatacagcccaaagcaagcctccag

SEQ ID NO: 24 - DNA, 151 bp, Artificial Sequence
  Synthetic Polynucleotide
tggaggcttgctgaaggctgtatgctgttgtcggggccggtttcggggccttagtgaagccacagatgtacggccccgaaaccggccccaggacacaaggcctgttactagcactcacatggaacaaatggccaccgtgggaggatgacaa

SEQ ID NO: 25 - DNA, 151 bp, Artificial Sequence
  Synthetic Polynucleotide
ttgtcatcctcccacggtggccatttgttccatgtgagtgctagtaacaggccttgtgtcctggggccggtttcggggccgtacatctgtggcttcactaaggccccgaaaccggccccgacaacagcatacagccttcagcaagcctcca

SEQ ID NO: 26 - DNA, 4145 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcggggccggtttcggggccttagtgaagccacagatgtacggccccgaaaccggccccaggacacaaggcctgttactagcactcacatggaacaaatggccaccgtgggaggatgacaatggaggcttgctgaaggctgtatgctgttgtcttggcctttcggttcctccagtgaagccacagatgggaggaaccgaaaggccaaaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 27 - DNA, 899 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagcaaaaaaattgtcatcctcccacggtggccatttgttccatgtgagtgctagtaacaggccttgtgtcctttggcctttcggttcctcccatctgtggcttcactggaggaaccgaaaggccaagacaacagcatacagccttcagcaagcctccagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaagcttggatctgatccctaggttctagaaccggtgaccaattgttaattaagtttaaaccctcgaggccgcaagcagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 28 - DNA, 2547 bp, Artificial Sequence
  Synthetic Polynucleotide
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcttggcctttcggttcctccagtgaagccacagatgggaggaaccgaaaggccaaaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccacccaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 29 - DNA, 19 bp, Artificial Sequence
  Synthetic Polynucleotide
tattagatctgatggccgc

SEQ ID NO: 30 - DNA, 20 bp, Artificial Sequence
  Synthetic Polynucleotide
ctccatcactaggggttcct

SEQ ID NO: 31 - DNA, 60 bp, Artificial Sequence
  Synthetic Polynucleotide
agctctgggtatttaagcccgagtgagcacgcagggtctccattttgaagcgggaggtta

SEQ ID NO: 32 - DNA, 145 bp, Artificial Sequence
  Synthetic Polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 33 - DNA, 24 bp, Artificial Sequence
  Synthetic Polynucleotide
gtgtactaggatcttttacttgaa

SEQ ID NO: 34 - DNA, 24 bp, Artificial Sequence
  Synthetic Polynucleotide
ttcaagtaaaagatcctagtacac

SEQ ID NO: 35 - DNA, 267 bp, Artificial Sequence
  Synthetic Polynucleotide
gtgatatcacaaggtcccagggctggggtcagaaattctctcccgagggaatgaagccacaggagccaagagcaggaggaccaaggccctggcgaaggccgtggcctcgttcaagtaaaagatcctagtacagtgcaggtcccaatgtgtactaggatcttttacttgaacggggacgccggcatccgggctcaggacccccctctctgccagaggcaccaacaccagagttcacaaatcagtctcctgccctttgcatgtagcaaa

SEQ ID NO: 36 - DNA, 267 bp, Artificial Sequence
  Synthetic Polynucleotide
tttgctacatgcaaagggcaggagactgatttgtgaactctggtgttggtgcctctggcagagaggggggtcctgagcccggatgccggcgtccccgttcaagtaaaagatcctagtacacattgggacctgcactgtactaggatcttttacttgaacgaggccacggccttcgccagggccttggtcctcctgctcttggctcctgtggcttcattccctcgggagagaatttctgaccccagccctgggaccttgtgatatcac

SEQ ID NO: 37 - DNA, 19 bp, Artificial Sequence
  Synthetic Polynucleotide
atgcatccatattcttcct

SEQ ID NO: 38 - DNA, 19 bp, Artificial Sequence
  Synthetic Polynucleotide
aggaagaatatggatgcat

SEQ ID NO: 39 - DNA, 148 bp, Artificial Sequence
  Synthetic Polynucleotide
tggaggcttgctgaaggctgtatgctgttgtcatgcatccatattcttcctagtgaagccacagatgaggaagaatatggatgcataggacacaaggcctgttactagcactcacatggaacaaatggccaccgtgggaggatgacaa

SEQ ID NO: 40 - DNA, 19 bp, Artificial Sequence
  Synthetic Polynucleotide
ttatttactttctctgcac

SEQ ID NO: 41 - DNA, 19 bp, Artificial Sequence
  Synthetic Polynucleotide
gtgcagagaaagtaaataa

SEQ ID NO: 42 - DNA, 148 bp, Artificial Sequence
  Synthetic Polynucleotide
tggaggcttgctgaaggctgtatgctgttgtcttatttactttctctgcacagtgaagccacagatggtgcagagaaagtaaataaaggacacaaggcctgttactagcactcacatggaacaaatggccaccgtgggaggatgacaa

SEQ ID NO: 43 - DNA, 151 bp, Artificial Sequence; identical to SEQ ID NO: 24
  Synthetic Polynucleotide

SEQ ID NO: 44 - DNA, 151 bp, Artificial Sequence; identical to SEQ ID NO: 24
  Synthetic Polynucleotide

SEQ ID NO: 45 - DNA, 296 bp, Artificial Sequence
  Synthetic Polynucleotide
ttgtcatcctcccacggtggccatttgttccatgtgagtgctgataaagggccttgtgtcctgtctggttaatctttatcaggttcagtcagaggccaaaaacctgataaagattaaccagacagcatacagcccaaagcaagcctccagttgtcatcctcccacggtggccatttgttccatgtgagtgctgataaagggccttgtgtcctgcggttgcggtgcctgcgcctcagtcagaggccaaaaggcgcaggcaccgcaaccgcagcatacagcccaaagcaagcctccag

SEQ ID NO: 46 - DNA, 296 bp, Artificial Sequence
  Synthetic Polynucleotide
ctggaggcttgctttgggctgtatgctgcggttgcggtgcctgcgccttttggcctctgactgaggcgcaggcaccgcaaccgcaggacacaaggccctttatcagcactcacatggaacaaatggccaccgtgggaggatgacaactggaggcttgctttgggctgtatgctgtctggttaatctttatcaggtttttggcctctgactgaacctgataaagattaaccagacaggacacaaggccctttatcagcactcacatggaacaaatggccaccgtgggaggatgacaa

SEQ ID NO: 47 - DNA, 297 bp, Artificial Sequence
  Synthetic Polynucleotide
ttgtcatcctcccacggtggccatttgttccatgtgagtgctgataaagggccttgtgtcctgggttgtttccctccttgttttcagtcagaggccaaaataaacaaggagggaaacaacccagcatacagcccaaagcaagcctccagttgtcatcctcccacggtggccatttgttccatgtgagtgctgataaagggccttgtgtcctggctgcggttgcggtgcctgctcagtcagaggccaaaagcaggcaccgcaaccgcagccagcatacagcccaaagcaagcctccag

SEQ ID NO: 48 - DNA, 297 bp, Artificial Sequence
  Synthetic Polynucleotide
ctggaggcttgctttgggctgtatgctggctgcggttgcggtgcctgcttttggcctctgactgagcaggcaccgcaaccgcagccaggacacaaggccctttatcagcactcacatggaacaaatggccaccgtgggaggatgacaactggaggcttgctttgggctgtatgctgggttgtttccctccttgtttattttggcctctgactgaaaacaaggagggaaacaacccaggacacaaggccctttatcagcactcacatggaacaaatggccaccgtgggaggatgacaa

SEQ ID NO: 49 - DNA, 294 bp, Artificial Sequence
  Synthetic Polynucleotide
ttgtcatcctcccacggtggccatttgttccatgtgagtgctgataaagggccttgtgtcctgtttcttctggttaatctttatcagtcagaggccaaaataaagattaaccagaagaaacagcatacagcccaaagcaagcctccagttgtcatcctcccacggtggccatttgttccatgtgagtgctgataaagggccttgtgtcctgcggtgcctgcgcccgcggctcagtcagaggccaaaagccgcgggcgcaggcaccgcagcatacagcccaaagcaagcctccag

SEQ ID NO: 50 - DNA, 294 bp, Artificial Sequence
  Synthetic Polynucleotide
ctggaggcttgctttgggctgtatgctgcggtgcctgcgcccgcggcttttggcctctgactgagccgcgggcgcaggcaccgcaggacacaaggccctttatcagcactcacatggaacaaatggccaccgtgggaggatgacaactggaggcttgctttgggctgtatgctgtttcttctggttaatctttattttggcctctgactgataaagattaaccagaagaaacaggacacaaggccctttatcagcactcacatggaacaaatggccaccgtgggaggatgacaa

SEQ ID NO: 51 - DNA, 1446 bp, Artificial Sequence; identical to SEQ ID NO: 3
  Synthetic Polynucleotide

SEQ ID NO: 52 - DNA, 3994 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcatgcatccatattcttcctagtgaagccacagatgaggaagaatatggatgcataggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 53 - DNA, 3997 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcggggccggtttcggggccttagtgaagccacagatgtacggccccgaaaccggccccaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 54 - DNA, 3994 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcttatttactttctctgcacagtgaagccacagatggtgcagagaaagtaaataaaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 55 - DNA, 902 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagcaaaaaaattgtcatcctcccacggtggccatttgttccatgtgagtgctagtaacaggccttgtgtcctggggccggtttcggggccgtacatctgtggcttcactaaggccccgaaaccggccccgacaacagcatacagccttcagcaagcctccagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaagcttggatctgatccctaggttctagaaccggtgaccaattgttaattaagtttaaaccctcgaggccgcaagcagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 56 - DNA, 2550 bp, Artificial Sequence
  Synthetic Polynucleotide
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcggggccggtttcggggccttagtgaagccacagatgtacggccccgaaaccggccccaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccacccaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 57 - DNA, 4142 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtctggaggcttgctttgggctgtatgctgcggttgcggtgcctgcgccttttggcctctgactgaggcgcaggcaccgcaaccgcaggacacaaggccctttatcagcactcacatggaacaaatggccaccgtgggaggatgacaactggaggcttgctttgggctgtatgctgtctggttaatctttatcaggtttttggcctctgactgaacctgataaagattaaccagacaggacacaaggccctttatcagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 58 - DNA, 4143 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtctggaggcttgctttgggctgtatgctggctgcggttgcggtgcctgcttttggcctctgactgagcaggcaccgcaaccgcagccaggacacaaggccctttatcagcactcacatggaacaaatggccaccgtgggaggatgacaactggaggcttgctttgggctgtatgctgggttgtttccctccttgtttattttggcctctgactgaaaacaaggagggaaacaacccaggacacaaggccctttatcagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 59 - DNA, 4140 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtctggaggcttgctttgggctgtatgctgcggtgcctgcgcccgcggcttttggcctctgactgagccgcgggcgcaggcaccgcaggacacaaggccctttatcagcactcacatggaacaaatggccaccgtgggaggatgacaactggaggcttgctttgggctgtatgctgtttcttctggttaatctttattttggcctctgactgataaagattaaccagaagaaacaggacacaaggccctttatcagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 60 - DNA, 3793 bp, Artificial Sequence
  Synthetic Polynucleotide
tctcattagaagtgaggcggggccggccaaatcgaatggacaccgggtaattagcagggttacccagatactccagcacctctttcccgtcggccgtgtacctgccattcacgtccatgccattgatggccagcactgcatgacccactgcagaggtgaagctaacggtcagcgaaggtgcagcccggggattccgccgaggggacaagggacccgacacaaccccttttcccccaaccccgcacctacaaccagcccacttctacagcactggggccctcccacccccgcacccgccacgggcccgagcctagcccacctcggatgccgtcccgctggccgaaagcaaccaacacacgctcatcgtgtagcttgagcagcagatccagcggataactgaaagttttctcagcctcagcccgtggcgcgtagctgtccaactggtaaatcaagccgccagctttgttcaccacatacacactaaaaatcgccatcgctgccttgccgctcggaaactggtattcagcctctacccgacggcccctccccggaaccgcatcacagcacttgccgccggccccaccccagcctcctcctcctcctcctcctcctcccgcgccccccgtgcagccacctgctgcacttgcgcactgggagcgacacgctcgggcataagtagtgccgaaaagttagctgccgagacctggtggattgcttttcgtttatcagtgcaggaaaacagcgctatagtactgcgtcacaactagcgcagactccggcagtatttaggcggtgcggcttgggaactagaatccacttcctgtcttccgcctcaggctagagggcgagcgcttcgccgtgggacttcttctgcctggctccgcctcttgccccggaagtactcacagcggacggtggtttttgggcccgtttctgagcagcgcttcctttttgtccgacatcttgacgaggctgcggtgtctgctgctattctccgagcttcgcaatggtaagcttcaggggtgtgaagtcgccggcgttcttgggtttgaggactcagtggggagagccttcggcgggagcgctccttggcctgccggcctcggttgcagggcgggcgcggttattgcttggcccatgtgctctggtggtggagtttgcgggggctgagggcgcagtattaggggactttggcgctatttgaggacctggttgcattcccgctgccctcctacagccgcctaaggacgacaagaagaagaaggacgctggaaagtcggccaagaaagacaaagacccagtgaacaaatccgggggcaaggccaaaaagaaggtagaaataagacctctctgaaagagactaggggtaactctctcgtaatcctctagtaataggtaacttgtatagtaagtggtttttcaggtgtagatttctagagtcaaaatgtgagagtttatcttcccgtcaccactcgttctttttcccattaggatcatgaaaatgggtctgttgtgcgaagtgtctgccgctgtgcctgctgtgttatttttaactgatctagtggggctcggcccctgtttgaaggccaaaaacgtgtcggtgttttttttttgtttttgttttagtaatgtgtaatttatccttgataacggtggaacagatttctctgacgcagattactcgagagggaaagggtgcttctgccagaaatactaacttgtttctgttttgttttggtgagcagaagtggtccaaaggcaaagttcgggacaagctcaataacttagtcttgtttgacaaagctacctatgataaactctgtaaggaagttcccaactataaacttataaccccagctgtggtctctgagagactgaagattcgaggctccctggccagggcagcccttcaggagctccttagtaaaggtgaggggtgtatcctacatgtgtgtttttgtaggttaaattgtcttgaccatgttaagcatcttcagtggttttgctggaaaagcagaattaaaaaaaaaaagcgtggcttgaccattggctgttagtaatgtaattctgacgtcttactcctgatcctgagatgaattctcagggttcttagccacttttgtgccgtggaccctgtggcagtttagtgaagcccaaggatcttttatgtttcgagtaaatggatgcatagaattacagggacaaccgtttttgaaataattagattactattttgaaacaactttgaaaatgtttaaaacctttatggtaaatattttgttgatgtattaaattttaaaaccagaaatttagtacggtctactcagtagtatggtctgattaccataattccacaataataaggctcagctaactatagtgactgaacgtctataattctagcactttgggaggccaaggcgggtgaatcaacggaggtcaggagttaaagaccagcctggccaatatggtgaaaacctgctctactgaaagttagctggacgtgggggcacacgtctgtaatcccagctactcaggatgctgaggcatgaggatcccttgaacccaggagatggaggtggcagtgagccgagatgacaccactgcactccagccttagtgacagcaaaagactgtctcagaaagggggggggggtggaagataatggagccctaatttaaaggaaaagtaaggatagatgatccgttaaaaacttggattctcggttaccgaacgtcagattaagcaattctggagccaggtgcagtggtacccttgtatttctagctacttgggaggccaaagcaggaggatcatttgagccaaggagttttaagaccattctgggcacctctgagagaactctgtctttttgttttccttttctttaaatagagatgcggttttgccatgttgcccaggctggtctcctgggctcaagagatccacctgtccaaagtgctgggattacaggcatgagcctctgcacccggccaaaacaaaccttactagagtctcattctgttgcccaggttggagtgcggaggggcagtcttggctcaatgcaaccaccaattcctgggttcaggtggtcctcacctcagcttcccaagtagctggaattacaagcatgtgccaccatgcccagctaatttttgtatttttggtagagatggggtttcaccttgttggccaggctggtgtgcaactccttacctcaagctatctgcccgtctccacctcccaaagcagtgggattataagcatgagccaccgcgcccagccaaaaaccttactagtttctattgtagcatctgttaagcatctcatcgtgctattctctccccctaggacttatcaaactggtttcaaagcacagagctcaagtaatttacaccagaaataccaagggtggagatgctccagctgctggtgaagatgcatgaataggtgagtaggaatgtgtgggctcatggtgtaggaggtagatacaaagctttatggttctgattcttttaatttttttttacaggtccaaccagctgtacatttggaaaaataaaactttattaaatcaaatgaatgagtatgtctgtttcctaagaaagacaatgataaagaatttggtggaaggtataataggggtttgttgactttgcttttagcctcatggtagttggtagagagcatgattagcttttttctgtatgtgactgcttcttcattgctgcagcttcagttttgaattgatgtctgaaaggaaataaagggttaacacgatgatgaagggtg

SEQ ID NO: 61 - DNA, 3996 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtctggaggcttgctttgggctgtatgctgataccttccaccaaattctttattttggcctctgactgataaagaattgtggaaggtatcaggacacaaggccctttatcagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 62 - DNA, 3994 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtctggaggcttgctttgggctgtatgctgataagtcctttactaaggagcttttggcctctgactgagctccttgtaaggacttatcaggacacaaggccctttatcagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgagcaccctgtgccccccccccagccccgccgtggccaagaccgagatcgccctgagcggcaagagccccctgctggccgccaccttcgcctactgggacaacatcctgggcccccgcgtgcgccacatctgggcccccaagaccgagcaggtgctgctgagcgacggcgagatcaccttcctggccaaccacaccctgaacggcgagatcctgcgcaacgccgagagcggcgccatcgacgtgaagttcttcgtgctgagcgagaagggcgtgatcatcgtgagcctgatcttcgacggcaactggaacggcgaccgcagcacctacggcctgagcatcatcctgccccagaccgagctgagcttctacctgcccctgcaccgcgtgtgcgtggaccgcctgacccacatcatccgcaagggccgcatctggatgcacaaggagcgccaggagaacgtgcagaagatcatcctggagggcaccgagcgcatggaggaccagggccagagcatcatccccatgctgaccggcgaggtgatccccgtgatggagctgctgagcagcatgaagagccacagcgtgcccgaggagatcgacatcgccgacaccgtgctgaacgacgacgacatcggcgacagctgccacgagggcttcctgctgaacgccatcagcagccacctgcagacctgcggctgcagcgtggtggtgggcagcagcgccgagaaggtgaacaagatcgtgcgcaccctgtgcctgttcctgacccccgccgagcgcaagtgcagccgcctgtgcgaggccgagagcagcttcaagtacgagagcggcctgttcgtgcagggcctgctgaaggacagcaccggcagcttcgtgctgcccttccgccaggtgatgtacgccccctaccccaccacccacatcgacgtggacgtgaacaccgtgaagcagatgcccccctgccacgagcacatctacaaccagcgccgctacatgcgcagcgagctgaccgccttctggcgcgccaccagcgaggaggacatggcccaggacaccatcatctacaccgacgagagcttcacccccgacctgaacatcttccaggacgtgctgcaccgcgacaccctggtgaaggccttcctggaccaggtgttccagctgaagcccggcctgagcctgcgcagcaccttcctggcccagttcctgctggtgctgcaccgcaaggccctgaccctgatcaagtacatcgaggacgacacccagaagggcaagaagcccttcaagagcctgcgcaacctgaagatcgacctggacctgaccgccgagggcgacctgaacatcatcatggccctggccgagaagatcaagcccggcctgcacagcttcatcttcggccgccccttctacaccagcgtgcaggagcgcgacgtgctgatgaccttctaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

(19) United States
(12) Patent Application Publication
(10) Pub. No.: US 2021/0261981 A1
(43) Pub. Date: Aug. 26, 2021

(54) GENE THERAPIES FOR NEURODEGENERATIVE DISEASES

(71) Applicant: Prevail Therapeutics, Inc., New York, NY (US)

(72) Inventors: Asa Abeliovich, New York, NY (US); Laura Heckman, New York, NY (US); Herve Rhinn, New York, NY (US)

(73) Assignee: Prevail Therapeutics, Inc., New York, NY (US)

(21) Appl. No.: 16/758,077

(22) PCT Filed: Oct. 23, 2018
(86) PCT No.: PCT/US2018/057187

§ 371 Date: Apr. 22, 2020

Related U.S. Application Data
(60) Provisional application No. 62/742,723, filed on Oct. 8, 2018.
(60) Provisional application No. 62/575,795, filed on Oct. 23, 2017.

(51) Int. Cl. C12N 15/86; C12N 15/113; A61K 9/00

(52) U.S. Cl. CPC C12N 15/86; C12N 15/1138; A61K 9/0085; C12N 2320/32; C12N 2330/51; C12N 2710/14043; C12N 2310/14; C12N 2750/14143

(57) ABSTRACT

The disclosure relates, in some aspects, to compositions and methods for treatment of neurodegenerative diseases (e.g., amyotrophic lateral sclerosis (ALS) and/or frontotemporal dementia (FTD), Alzheimer's disease, Gaucher disease, Parkinson's disease, Lewy body dementia, or a lysosomal storage disease). In some embodiments, the disclosure provides expression constructs comprising a transgene encoding one or more inhibitory nucleic acids (e.g., targeting C9orf72, TMEM106B, ATNX2, RPS25, etc.), wild-type C9orf72 protein or a portion thereof, or any combination of the foregoing. In some embodiments, the disclosure provides methods of ALS/FTD by administering such expression constructs to a subject in need thereof.

### RELATED APPLICATIONS

This application claims the benefit under 35 U.S.C. § 119(e) of U.S. provisional application Ser. Nos. 62/742,723, filed Oct. 8, 2018, entitled “GENE THERAPIES FOR NEURODEGENERATIVE DISEASE”, and 62/575,795, filed Oct. 23, 2017, entitled “GENE THERAPIES FOR NEURODEGENERATIVE DISEASE”, the entire contents of each of which are incorporated herein by reference.

### BACKGROUND

Amyotrophic lateral sclerosis (ALS) and frontotemporal dementia (FTD) are neurodegenerative diseases that are linked to expansion of a hexanucleotide repeat region in the C9orf72 gene in humans. Generally, pathology associated with expansion of the C9orf72 repeat region is caused by decreased expression of the C9orf72 protein and a gain of function due to accumulation of toxic RNA foci. Currently, treatment options for ALS/FTD are limited.

### SUMMARY

Aspects of the disclosure relate to compositions and methods useful for the treatment of neurodegenerative diseases, such as amyotrophic lateral sclerosis (ALS) and/or frontotemporal dementia (FTD), Alzheimer's disease, Gaucher disease, Parkinson's disease, Lewy body dementia, or a lysosomal storage disease. In some embodiments, methods and compositions described herein are useful for treating subject having ALS/FTD characterized by an expansion of the dipeptide repeat region of the C9orf72 gene.

In some aspects, the disclosure provides an isolated nucleic acid comprising an expression cassette encoding an inhibitory nucleic acid that inhibits expression or activity of C9orf72 and/or ataxin 2 (ATXN2) and/or ribosomal protein 25 (RPS25). In some embodiments, an inhibitory nucleic acid targeting ATXN comprises or consists of a sequence set forth in any one of SEQ ID NO: 10-25. In some embodiments, an inhibitory nucleic acid targeting C9orf72 comprises or consists of a sequence set forth in any one of SEQ ID NOs: 37-50.

In some aspects, the disclosure provides an isolated nucleic acid comprising an expression cassette encoding a codon-optimized C9orf72 protein (or a portion thereof). In some embodiments, a codon-optimized C9orf72 protein comprises the amino acid sequence set forth in SEQ ID NO: 4. In some embodiments, a codon-optimized C9orf72 protein is encoded by a nucleic acid having the sequence set forth in SEQ ID NO: 51.

In some aspects, the disclosure provides an isolated nucleic acid comprising an expression cassette encoding an inhibitory nucleic acid that inhibits expression or activity of C9orf72 and/or ATXN2 and/or RPS25, and a wild-type C9orf72 protein (e.g., a C9orf72 protein lacking a pathogenic dipeptide repeat expansion). In some embodiments, a wild-type C9orf72 protein is encoded by SEQ ID NO: 3, or a portion thereof. In some embodiments, a wild-type C9orf72 protein comprises or consists of the sequence set forth in SEQ ID NO: 4, or a portion thereof.

In some aspects, the disclosure provides an isolated nucleic acid comprising an expression cassette encoding a first inhibitory nucleic acid that inhibits expression or activity of C9orf72 and, a second inhibitory nucleic acid that inhibits expression or activity of ataxin 2 (ATXN2). In some aspects, the disclosure provides an isolated nucleic acid comprising an expression cassette encoding a first inhibitory nucleic acid that inhibits expression or activity of C9orf72 and, a second inhibitory nucleic acid that inhibits expression or activity of Transmembrane Protein 106B (TMEM106B). In some aspects, the disclosure provides an isolated nucleic acid comprising an expression cassette encoding a first inhibitory nucleic acid that inhibits expression or activity of C9orf72 and, a second inhibitory nucleic acid that inhibits expression or activity of RPS25. In some embodiments, an isolated nucleic acid further comprises a nucleic acid sequence encoding a wild-type C9orf72 protein (e.g., as set forth in SEQ ID NO: 3).

In some aspects, the disclosure provides an isolated nucleic acid comprising an expression cassette encoding an inhibitory nucleic acid that inhibits expression or activity of C9orf72 and, a β-glucocerebrosidase (GBA) protein. In some embodiments, the GBA protein is a GBA1 protein (e.g., a protein encoded by the GBA1 gene or a portion thereof). In some aspects, the disclosure provides an isolated nucleic acid comprising an expression cassette encoding an inhibitory nucleic acid that inhibits expression or activity of ATXN2 and, a β-glucocerebrosidase (GBA) protein. In some embodiments, the GBA protein is a GBA1 protein (e.g., a protein encoded by the GBA1 gene or a portion thereof). In some aspects, the disclosure provides an isolated nucleic acid comprising an expression cassette encoding an inhibitory nucleic acid that inhibits expression or activity of TMEM106B and, a β-glucocerebrosidase (GBA) protein. In some embodiments, the GBA protein is a GBA1 protein (e.g., a protein encoded by the GBA1 gene or a portion thereof).

In some embodiments, an inhibitory nucleic acid (e.g., a first inhibitory nucleic acid, second inhibitory nucleic acid, third inhibitory nucleic acid, etc.) binds to a nucleic acid encoding a dipeptide-repeat region of C9orf72 (e.g., a C9orf72 mRNA transcript that comprises a dipeptide-repeat region). In some embodiments, a dipeptide-repeat region comprises one or more GGGGCC repeats, or one or more CCCCGG repeats (e.g., the dipeptide repeat region of C9orf72). In some embodiments, a dipeptide-repeat region comprises 23 or more (for example, any integer between 23 and 10,000, e.g., 24, 25, 30, 50, 100, 1000, 5000, or 10,000) GGGGCC repeats (e.g., the sense strand dipeptide repeat region of C9orf72), or 23 or more(for example, any integer between 23 and 10,000, e.g., 24, 25, 30, 50, 100, 1000, 5000, or 10,000) CCCCGG repeats (e.g., the antisense strand dipeptide repeat region of C9orf72).

In some embodiments, an inhibitory nucleic acid binds to a nucleic acid encoding a region of C9orf72 that is not a dipeptide-repeat region (e.g., a portion of the nucleic acid that is outside of the C9orf72 dipeptide-repeat region). In some embodiments, an inhibitory nucleic acid binds to an isolated nucleic acid sequence that is within between 1 nucleic acid (e.g., adjacent to a dipeptide-repeat region) and about 500 nucleic acids of a dipeptide-repeat region. In some embodiments, an inhibitory nucleic acid targets an intronic region of a gene encoding C9orf72 protein.

In some embodiments, an inhibitory nucleic acid (e.g., a first inhibitory nucleic acid, second inhibitory nucleic acid, third inhibitory nucleic acid, etc.) binds to a nucleic acid sequence encoding ATXN2 (e.g., an ATXN2 mRNA transcript), for example as set forth in SEQ ID NO: 9. In some embodiments, an inhibitory nucleic acid targeting ATXN2 binds to an untranslated region (e.g., 5′UTR, 3′UTR, etc.) of a nucleic acid sequence encoding ATXN2.

In some embodiments, an inhibitory nucleic acid (e.g., a first inhibitory nucleic acid, second inhibitory nucleic acid, third inhibitory nucleic acid, etc.) binds to a nucleic acid sequence encoding TMEM106B (e.g., a TMEM106B mRNA transcript), for example as set forth in SEQ ID NO: 7. In some embodiments, an inhibitory nucleic acid targeting TMEM106B binds to an untranslated region (e.g., 5′UTR, 3′UTR, etc.) of a nucleic acid sequence encoding TMEM106B.

In some embodiments, an inhibitory nucleic acid (e.g., a first inhibitory nucleic acid, second inhibitory nucleic acid, third inhibitory nucleic acid, etc.) binds to a nucleic acid sequence encoding RPS25 (e.g., a RPS25 mRNA transcript), for example as set forth in SEQ ID NO: 60. In some embodiments, an inhibitory nucleic acid targeting RPS25 binds to an untranslated region (e.g., 5′UTR, 3′UTR, etc.) of a nucleic acid sequence encoding RPS25.

In some embodiments, an inhibitory nucleic acid (e.g., a first inhibitory nucleic acid and/or a second inhibitory nucleic acid) is a siRNA, shRNA, miRNA, and dsRNA. In some embodiments, an miRNA is an artificial miRNA (amiRNA) that comprises an inhibitory nucleic acid sequence flanked by miRNA scaffold sequence, for example a miR-155 scaffold sequence.

In some embodiments, an inhibitory nucleic acid (e.g., a first inhibitory nucleic acid and/or a second inhibitory nucleic acid) is located in an untranslated region of the expression construct. In some embodiments, an untranslated region is an intron, a 5′ untranslated region (5′UTR), or a 3′ untranslated region (3′UTR).

In some embodiments, an isolated nucleic acid comprises one or more promoters. In some embodiments a promoter is a RNA pol III promoter (e.g., U6 or H1), RNA pol II promoter, chicken-beta actin (CBA) promoter, CAG promoter, CD68 promoter, or JeT promoter.

In some embodiments, an expression construct is flanked by two adeno-associated virus (AAV) inverted terminal repeat (ITR) sequences. In some embodiments, one of the ITR sequences flanking an expression construct lacks a functional terminal resolution site.

The disclosure relates, in some aspects, to rAAV vectors comprising an ITR having a modified “D” region (e.g., a D sequence that is modified relative to wild-type AAV2 ITR, SEQ ID NO: 32). In some embodiments, the ITR having the modified D region is the 5′ ITR of the rAAV vector. In some embodiments, a modified “D” region comprises an “S” sequence, for example as set forth in SEQ ID NO: 29. In some embodiments, the ITR having the modified “D” region is the 3′ ITR of the rAAV vector. In some embodiments, a modified “D” region comprises a 3′ITR in which the “D” region is positioned at the 3′ end of the ITR (e.g., on the outside or terminal end of the ITR relative to the transgene insert of the vector). In some embodiments, a modified “D” region comprises a sequence as set forth in SEQ ID NO: 29 or 30.

In some embodiments, an isolated nucleic acid (e.g., an rAAV vector) comprises a TRY region. In some embodiments, a TRY region comprises the sequence set forth in SEQ ID NO: 31.

In some embodiments, an isolated nucleic acid comprises the sequence (or encodes an amino acid sequence) set forth in any one of SEQ ID NOs: 1-62, or a portion thereof.

In some aspects, the disclosure provides a vector comprising an isolated nucleic acid as described by the disclosure. In some embodiments, a vector is a plasmid, or a viral vector. In some embodiments, a viral vector is a recombinant adeno-associated virus vector (rAAV) (e.g., a transgene comprising an isolated nucleic acid sequence encoding one or more inhibitory nucleic acids and/or an isolated nucleic acid encoding one or more proteins, such as wild-type C9orf72 and/or GBA1, flanked by AAV ITRs) or a Baculovirus vector. In some embodiments, an rAAV vector is single-stranded (e.g., single-stranded DNA).

In some aspects, the disclosure provides a composition comprising an isolated nucleic acid or a vector as described by the disclosure. In some embodiments, a composition further comprises a pharmaceutically acceptable carrier.

In some aspects, the disclosure provides a host cell comprising an isolated nucleic acid or a vector as described by the disclosure. In some embodiments, a host cells is a eukaryotic cell (e.g., mammalian cell, insect cell, etc.) or a prokaryotic cell (e.g., bacterial cell).

In some aspects, the disclosure provides a recombinant adeno-associated virus (rAAV) comprising a capsid protein and an isolated nucleic acid or vector as described by the disclosure. In some embodiments, a capsid protein is capable of crossing the blood-brain barrier. In some embodiments, a capsid protein is an AAV9 capsid protein, an AAVrh.10 capsid protein, or an AAV-PHP.B capsid protein. In some embodiments, a rAAV transduces neuronal cells and/or non-neuronal cells of the central nervous system (CNS).

In some aspects, the disclosure provides a method for treating a subject having or suspected of having a neurodegenerative disorder (e.g., amyotrophic lateral sclerosis (ALS) and/or frontotemporal dementia (FTD), Alzheimer's disease, Gaucher disease, Parkinson's disease, Lewy body dementia, or a lysosomal storage disease), the method comprising administering to the subject an isolated nucleic, a vector, a composition, or a rAAV as described by the disclosure.

In some embodiments, administration comprises direct injection to the CNS of a subject. In some embodiments, direct injection to the CNS comprises direct injection to the cerebrospinal fluid (CSF) of a subject, for example intracisternal injection, intraventricular injection, intralumbar injection, or any combination thereof. In some embodiments, direct injection is intracerebral injection, intraparenchymal injection, intrathecal injection, intra-cisterna magna injection, or any combination thereof. In some embodiments, direct injection comprises convection enhanced delivery (CED).

In some embodiments, the subject is a mammal, for example a human subject. In some embodiments, a subject is characterized by having between about 30 and about 5000 (e.g., any integer between 30 and 5000, inclusive) GGGGCC dipeptide repeats and/or between about 30 and 5000 (e.g., any integer between 30 and 5000, inclusive) CCCCGG repeats. In some embodiments, a subject is characterized by having more than 5000 GGGGCC dipeptide repeats and/or CCCCGG repeats.

### BRIEF DESCRIPTION OF THE DRAWINGS

FIG. 1 is a schematic depicting one embodiment of a plasmid comprising an rAAV vector that includes an expression construct encoding an inhibitory nucleic acid targeting the repeat expansion of C9orf72, an inhibitory nucleic acid targeting Transmembrane Protein 106B (TMEM106B), and a wild-type C9orf72 coding sequence. The rAAV vector further comprises AAV inverted terminal repeats flanking the expression construct.

FIG. 2 is a schematic depicting one embodiment of a plasmid comprising an rAAV that includes an expression construct encoding an inhibitory nucleic acid targeting the repeat expansion of C9orf72 and a β-glucocerebrosidase (GBA1) coding sequence. The rAAV vector further comprises AAV inverted terminal repeats flanking the expression construct.

FIG. 3 is a schematic depicting one embodiment of a plasmid comprising an rAAV vector that includes an expression construct encoding an inhibitory nucleic acid targeting the repeat expansion of C9orf72 and a wild-type C9orf72 coding sequence. The rAAV vector further comprises AAV inverted terminal repeats flanking the expression construct.

FIG. 4 is a schematic depicting one embodiment of a plasmid comprising an rAAV vector that includes an expression construct encoding an inhibitory nucleic acid targeting ATXN2 (e.g., the gene encoding ATNX2) operably linked to a pol III (H1) promoter. The rAAV vector further comprises AAV inverted terminal repeats flanking the expression construct. The “D” sequence of the 3′UTR is located in an “outside” position.

FIG. 5 is a schematic depicting one embodiment of a plasmid comprising an rAAV vector that includes an expression construct encoding an inhibitory nucleic acid targeting ATXN2 (e.g., the gene encoding ATNX2) operably linked to a pol II (CBA) promoter. The rAAV vector further comprises AAV inverted terminal repeats flanking the expression construct. The “D” sequence of the 3′UTR is located in an “outside” position.

FIG. 6 is a schematic depicting one embodiment of a plasmid comprising an expression construct encoding an inhibitory nucleic acid targeting ATXN2 (e.g., the gene encoding ATNX2) operably linked to a pol II (CBA) promoter.

FIG. 7 is a schematic depicting one embodiment of a plasmid comprising an expression construct encoding two inhibitory nucleic acids, each one targeting ATXN2 (e.g., the gene encoding ATNX2) operably linked to a pol II (CBA) promoter.

FIG. 8 is a schematic depicting one embodiment of a plasmid comprising an expression construct encoding an inhibitory nucleic acid targeting ATXN2 (e.g., the gene encoding ATNX2) operably linked to a pol II (CBA) promoter.

FIG. 9 is a schematic depicting one embodiment of a plasmid comprising an rAAV vector that includes an expression construct encoding an inhibitory nucleic acid targeting ATXN2 (e.g., the gene encoding ATNX2) operably linked to a pol II (CBA) promoter and a codon-optimized nucleic acid sequence encoding a wild-type C9orf72 protein. The rAAV vector further comprises AAV inverted terminal repeats flanking the expression construct. The “D” sequence of the 3′UTR is located in an “outside” position.

FIG. 10 is a schematic depicting one embodiment of a plasmid comprising an expression construct encoding an inhibitory nucleic acid targeting C9orf72 operably linked to a pol II (CBA) promoter and a codon-optimized nucleic acid sequence encoding a wild-type C9orf72 protein.

FIG. 11 is a schematic depicting one embodiment of a plasmid comprising an expression construct encoding an inhibitory nucleic acid targeting C9orf72 operably linked to a pol II (CBA) promoter and a codon-optimized nucleic acid sequence encoding a wild-type C9orf72 protein.

FIG. 12 is a schematic depicting one embodiment of a plasmid comprising an expression construct encoding an inhibitory nucleic acid targeting C9orf72 operably linked to a pol II (CBA) promoter and a codon-optimized nucleic acid sequence encoding a wild-type C9orf72 protein.

FIG. 13 is a schematic depicting one embodiment of a plasmid comprising an expression construct encoding an inhibitory nucleic acid targeting C9orf72 operably linked to a RNA pol III (e.g., H1) promoter.

FIG. 14 is a schematic depicting one embodiment of a plasmid comprising an expression construct encoding an inhibitory nucleic acid targeting C9orf72 operably linked to a pol II (CBA) promoter.

FIG. 15 is a schematic depicting one embodiment of a plasmid comprising an expression construct encoding two inhibitory nucleic acids targeting C9orf72 operably linked to a pol II (CBA) promoter and a codon-optimized nucleic acid sequence encoding a wild-type C9orf72 protein.

FIG. 16 is a schematic depicting one embodiment of a plasmid comprising an expression construct encoding two inhibitory nucleic acids targeting C9orf72 operably linked to a pol II (CBA) promoter and a codon-optimized nucleic acid sequence encoding a wild-type C9orf72 protein.

FIG. 17 is a schematic depicting one embodiment of a plasmid comprising an expression construct encoding two inhibitory nucleic acids targeting C9orf72 operably linked to a pol II (CBA) promoter and a codon-optimized nucleic acid sequence encoding a wild-type C9orf72 protein.

FIG. 18 is a schematic depicting an rAAV vectors comprising a “D” region located on the “outside” of the ITR (e.g., proximal to the terminus of the ITR relative to the transgene insert or expression construct) (top) and a wild-type rAAV vectors having ITRs on the “inside” of the vector (e.g., proximal to the transgene insert of the vector).

FIGS. 19A-19B show representative data for in vitro C9orf72 expression and knockdown assays. FIG. 19A shows representative data indicating statistically significant silencing of endogenous C9orf72 by rAAV vectors. FIG. 19B shows representative data indicating statistically significant increase in wild-type C9orf72 expression after transfection with rAAV vectors.

FIG. 20 is a schematic depicting one embodiment of a plasmid comprising an expression construct encoding an inhibitory nucleic acid targeting RPS25 operably linked to a pol II (CBA) promoter and a codon-optimized nucleic acid sequence encoding a wild-type C9orf72 protein.

FIG. 21 is a schematic depicting one embodiment of a plasmid comprising an expression construct encoding an inhibitory nucleic acid targeting RPS25 operably linked to a pol II (CBA) promoter and a codon-optimized nucleic acid sequence encoding a wild-type C9orf72 protein.

### DETAILED DESCRIPTION

Aspects of the disclosure relate to compositions and methods for treatment of neurodegenerative diseases, such as ALS/FTD, Parkinson's disease, Alzheimer's disease, lysosomal storage diseases, and Lewy body dementia. The disclosure is based, in part, on expression constructs encoding ALS/FTD-associated gene products (e.g., C9orf72, ATXN2, TMEM106B, inhibitory nucleic acids targeting the foregoing genes, etc.), and combinations thereof in a subject. A gene product can be a protein, a fragment (e.g., portion) of a protein, an interfering nucleic acid that inhibits an ALS/FTD-associated gene, etc. In some embodiments, a gene product is a protein or a protein fragment encoded by a ALS/FTD-associated gene. In some embodiments, a gene product is an interfering nucleic acid (e.g., shRNA, siRNA, miRNA, amiRNA, etc.) that inhibits an ALS/FTD-associated gene.

An ALS/FTD-associated gene refers to a gene encoding a gene product that is genetically, biochemically or functionally associated with amyotrophic lateral sclerosis (ALS), frontotemporal dementia (FTD), or ALS and FTD (ALS/FTD). For example, individuals having more than 23 GGGGCC hexanucleotide repeats in the C9orf72 gene, have been observed to be have an increased risk of developing ALS/FTD compared to individuals that do not have a repeat region expansion. In some embodiments, an expression cassette described herein encodes a wild-type or non-mutant form of an ALS/FTD-associated gene (or coding sequence thereof). Generally, a “wild-type” or “non-mutant” form of a gene refers to a nucleic acid that encodes a protein associated with normal or non-pathogenic activity (e.g., a protein lacking a mutation or change, such as a repeat region expansion that results in onset or progression of a neurodegenerative disease). For example, in some embodiments, a wild-type C9orf72 protein comprises or consists of the sequence set forth in SEQ ID NO: 4.

### Isolated Nucleic Acids and Vectors

An isolated nucleic acid may be DNA or RNA. In some aspects, the disclosure provides isolated nucleic acids (e.g., rAAV vectors) encoding one or more inhibitory nucleic acids that target one or more ALS/FTD-associated gene, for example C9orf72 (e.g., a dipeptide-repeat region of C9orf72), ATXN2, TMEM106B, RPS25, etc. An inhibitory nucleic acid may target a sense strand of a gene (e.g., an mRNA transcribed from a gene), an antisense strand of a gene (e.g., an mRNA transcribed from a gene), or both a sense and an antisense strand of a gene (e.g., an mRNA transcribed from a gene).

Generally, an isolated nucleic acid as described herein may encode 1, 2, 3, 4, 5, 6, 7, 8, 9, 10, or more inhibitory nucleic acids (e.g., dsRNA, siRNA, shRNA, miRNA, amiRNA, etc.). In some embodiments, an isolated nucleic acid encodes more than 10 inhibitory nucleic acids. In some embodiments, each of the one or more inhibitory nucleic acids targets a different gene or a portion of a gene (e.g., a first miRNA targets a first target sequence of a gene and a second miRNA targets a second target sequence of the gene that is different than the first target sequence). In some embodiments, each of the one or more inhibitory nucleic acids targets the same target sequence of the same gene (e.g., an isolated nucleic acid encodes multiple copies of the same miRNA).

Aspects of the disclosure relate to an isolated nucleic acid comprising an expression construct encoding one or more interfering nucleic acids (e.g., dsRNA, siRNA, miRNA, amiRNA, etc.) that target an C9orf72 protein (e.g., a dipeptide-repeat region of a C9orf72 mRNA transcript). In some embodiments, a dipeptide-repeat region is encoded by five or more polymer units of the hexanucleotide repeat sequence GGGGCC (e.g., a region comprising 5, 6, 7, 8, 9, 10, 20, 30, 40, 50, 100, 200, 500, 1000, or more repeats of the GGGGCC repeat sequence).

Generally, C9orf72 protein refers to a protein found in the cytoplasm of neurons and presynaptic terminals that are thought to be involved as exchange factors for small GTPases such as Rab. In humans, C9orf72 gene is located on chromosome 9. In some embodiments, the C9orf72 gene encodes a peptide that is represented by NCBI Reference Sequence NP_060795.1. In some embodiments, a C9orf72 gene comprises the sequence set forth in SEQ ID NO: 3 or encodes the amino acid sequence set forth in SEQ ID NO: 4.

An inhibitory nucleic acid targeting C9orf72 may comprise a region of complementarity (e.g., a region of the inhibitory nucleic acid that hybridizes to the target gene, such as C9orf72, or a portion of the target gene for example a dipeptide repeat region of C9orf72 or a region outside of a dipeptide-repeat region) that is between 6 and 50 nucleotides in length. In some embodiments, an inhibitory nucleic acid comprises a region of complementarity with C9orf72 that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a C9orf72 sequence. In some embodiments, the C9orf72 sequence targeted (e.g., bound) by the inhibitory nucleic acid is between lnucleotide and 500 nucleotides (e.g., any integer between 1 and 500 nucleotides, inclusive) away (either 5′ or 3′ relative to) the dipeptide-repeat region of C9orf72. In some embodiments, an inhibitory nucleic acid targets an intronic region (e.g., non-protein coding region) of a gene encoding C9orf72 protein.

Aspects of the disclosure relate to an isolated nucleic acid comprising an expression construct encoding one or more interfering nucleic acids (e.g., dsRNA, siRNA, miRNA, amiRNA, etc.) that target an TMEM106B protein (e.g., the gene product of TMEM106B gene). TMEM106B protein refers to transmembrane protein 106B, which is a protein involved in dendrite morphogenesis and regulation of lysosomal trafficking. In humans, TMEM106B gene is located on chromosome 7. In some embodiments, the TMEM106B gene encodes a peptide that is represented by NCBI Reference Sequence NP_060844.2. In some embodiments, a TMEM106B gene comprises the sequence set forth in SEQ ID NO: 7 or encodes the amino acid sequence set forth in SEQ ID NO: 6.

An inhibitory nucleic acid targeting TMEM106B may comprise a region of complementarity (e.g., a region of the inhibitory nucleic acid that hybridizes to the target gene, such as TMEM106B) that is between 6 and 50 nucleotides in length. In some embodiments, an inhibitory nucleic acid comprises a region of complementarity with TMEM106B that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a TMEM106B sequence.

Aspects of the disclosure relate to an isolated nucleic acid comprising an expression construct encoding one or more interfering nucleic acids (e.g., dsRNA, siRNA, miRNA, amiRNA, etc.) that target an ATXN2 protein (e.g., the gene product of ATXN2 gene, also referred to as SCA2 gene). ATXN2 protein refers to ataxin 2, which is a protein involved in regulating mRNA translation through its interactions with the poly(A)-binding protein. In humans, ATXN2 gene is located on chromosome 12. In some embodiments, the ATXN2 gene encodes a peptide that is represented by NCBI Reference Sequence NP_002964.3. In some embodiments, an ATXN2 gene comprises the sequence set forth in SEQ ID NO: 9 or encodes an amino acid sequence set forth in SEQ ID NO: 8.

An inhibitory nucleic acid targeting ATXN2 may comprise a region of complementarity (e.g., a region of the inhibitory nucleic acid that hybridizes to the target gene, such as ATXN2) that is between 6 and 50 nucleotides in length. In some embodiments, an inhibitory nucleic acid comprises a region of complementarity with ATXN2 that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a ATXN2 sequence.

Aspects of the disclosure relate to an isolated nucleic acid comprising an expression construct encoding one or more interfering nucleic acids (e.g., dsRNA, siRNA, miRNA, amiRNA, etc.) that target an ribosomal protein s25 (RPS25) (e.g., the gene product of RPS25). RPS25 protein refers to a ribosomal protein which is a subunit of the s40 ribosome, a protein complex involved in protein synthesis. In humans, RPS25 gene is located on chromosome 11. In some embodiments, the RPS25 gene encodes a peptide that is represented by NCBI Reference Sequence NP_001019.1. In some embodiments, a RPS25 gene comprises the sequence set forth in SEQ ID NO: 60.

An inhibitory nucleic acid targeting RPS25 may comprise a region of complementarity (e.g., a region of the inhibitory nucleic acid that hybridizes to the target gene, such as RPS25) that is between 6 and 50 nucleotides in length. In some embodiments, an inhibitory nucleic acid comprises a region of complementarity with RPS25 that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a RPS25 sequence.

Aspects of the disclosure relate to expression constructs comprising a first gene product encoding one or more inhibitory nucleic acids (e.g., an inhibitory nucleic acid targeting the dipeptide repeat region of C9orf72, an inhibitory nucleic acid targeting a non-dipeptide repeat region of C9orf72, and/or an inhibitory nucleic acid targeting TMEM106B, and/or an inhibitory nucleic acid targeting ATXN2, and/or an inhibitory nucleic acid targeting RPS25, etc.) and a second gene product encoding a protein, such as a wild-type C9orf72 protein or a GBA protein.

In some embodiments, an isolated nucleic acid comprises an expression cassette encoding a first inhibitory nucleic acid that inhibits expression or activity of C9orf72 and, a second inhibitory nucleic acid that inhibits expression or activity of TMEM106B.

In some embodiments, an isolated nucleic acid comprises an expression cassette encoding a first inhibitory nucleic acid that inhibits expression or activity of C9orf72 and, a second inhibitory nucleic acid that inhibits expression or activity of ATXN2.

In some embodiments, an isolated nucleic acid comprises an expression cassette encoding a first inhibitory nucleic acid that inhibits expression or activity of C9orf72 and, a second inhibitory nucleic acid that inhibits expression or activity of RPS25.

In some embodiments, an isolated nucleic acid comprises an expression cassette encoding an inhibitory nucleic acid that inhibits expression or activity of C9orf72 and, a β-glucocerebrosidase (GBA) protein. In some embodiments, the GBA protein is a GBA1 protein (e.g., a protein encoded by the GBA1 gene or a portion thereof).

In some embodiments, an isolated nucleic acid comprises an expression cassette encoding an inhibitory nucleic acid that inhibits expression or activity of C9orf72 and, a wild-type C9orf72 protein (e.g., a C9orf72 protein lacking a pathogenic dipeptide repeat expansion). In some embodiments, a nucleic acid sequence encoding a wild-type C9orf72 protein or a portion thereof is a codon-optimized nucleic acid sequence. In some embodiments, a wild-type C9orf72 protein is encoded by the nucleic acids sequence set forth in SEQ ID NO: 3, or a portion thereof. In some embodiments, a wild-type C9orf72 protein comprises or consists of the sequence set forth in SEQ ID NO: 4, or a portion thereof. In some embodiments, an isolated nucleic acid encoding a codon-optimized C9orf72 comprises or consists of the sequence set forth in SEQ ID NO: 51.

A skilled artisan recognizes that the order of expression of a first gene product (e.g., a nucleic acid sequence encoding a C9orf72 protein or a GBA protein) and a second gene product (e.g., inhibitory RNA targeting C9orf72, ATXN2, TMEM106B, etc.) can generally be reversed (e.g., the inhibitory RNA is the first gene product and protein coding sequence is the second gene product). In some embodiments, a gene product is a fragment (e.g., portion) of a gene (e.g., C9orf72, TMEM106B, ATXN2, GBA1, etc.). A protein fragment may comprise about 50%, about 60%, about 70%, about 80% about 90% or about 99% of a protein (e.g., a C9orf72 protein, a GBA protein, etc.). In some embodiments, a protein fragment comprises between 50% and 99.9% (e.g., any value between 50% and 99.9%) of a C9orf72 protein or a GBA protein. In some embodiments, a gene product (e.g., an inhibitory RNA) hybridizes to portion of a target gene (e.g., is complementary to 5, 6, 7, 8, 9, 10, 11, 12, 13, 14, 15, 16, 17, 18, 19, 20, 21, or more contiguous nucleotides of a target gene, for example C9orf72, ATXN2, or TMEM106B).

In some embodiments, an expression construct is monocistronic (e.g., the expression construct encodes a single fusion protein comprising a first gene product and a second gene product). In some embodiments, an expression construct is polycistronic (e.g., the expression construct encodes two distinct gene products, for example two different proteins or protein fragments).

A polycistronic expression vector may comprise a one or more (e.g., 1, 2, 3, 4, 5, or more) promoters. Any suitable promoter can be used, for example, a constitutive promoter, an inducible promoter, an endogenous promoter, a tissue-specific promoter (e.g., a CNS-specific promoter), etc. In some embodiments, a promoter is a chicken beta-actin promoter (CBA promoter), a CAG promoter (for example as described by Alexopoulou et al. (2008) BMC Cell Biol. 9: 2; doi: 10.1186/1471-2121-9-2), a CD68 promoter, or a JeT promoter (for example as described by Tornøe et al. (2002) Gene 297(1-2): 21-32, or Karumuthil-Melethil et al. (2016) Human Gene Therapy 27(7): 509-521). In some embodiments, a promoter is a RNA pol II promoter or a RNA pol III promoter (e.g., U6, H1, etc.). In some embodiments, a promoter is operably-linked to a nucleic acid sequence encoding a first gene product, a second gene product, or a first gene product and a second gene product. In some embodiments, an expression cassette comprises one or more additional regulatory sequences, including but not limited to transcription factor binding sequences, intron splice sites, poly(A) addition sites, enhancer sequences, repressor binding sites, or any combination of the foregoing.

In some embodiments, a nucleic acid sequence encoding a first gene product and a nucleic acid sequence encoding a second gene product are separated by a nucleic acid sequence encoding an internal ribosomal entry site (IRES). Examples of IRES sites are described, for example, by Mokrejs et al. (2006) Nucleic Acids Res. 34(Database issue): D125-30. In some embodiments, a nucleic acid sequence encoding a first gene product and a nucleic acid sequence encoding a second gene product are separated by a nucleic acid sequence encoding a self-cleaving peptide. Examples of self-cleaving peptides include but are not limited to T2A, P2A, E2A, F2A, BmCPV 2A, and BmIFV 2A, and those described by Liu et al. (2017) Sci Rep. 7: 2193. In some embodiments, the self-cleaving peptide is a T2A peptide.

Pathologically, disorders such as ALS and FTD are associated with accumulation of protein aggregates composed largely of repeat-associated non-ATG (RAN) translated proteins derived from the C9orf72 gene. Accordingly, in some embodiments, isolated nucleic acids described herein comprise an inhibitory nucleic acid that reduces or prevents expression of C9orf72 protein (e.g., C9orf72 protein encoded by a gene having a pathogenic dipeptide-repeat expansion). A sequence encoding an inhibitory nucleic acid may be placed in an untranslated region (e.g., intron, 5′UTR, 3′UTR, etc.) of the expression construct.

In some embodiments, an inhibitory nucleic acid is positioned in an intron of an expression construct, for example in an intron upstream of the sequence encoding a first gene product. An inhibitory nucleic acid can be a double stranded RNA (dsRNA), siRNA, micro RNA (miRNA), artificial miRNA (amiRNA), or an RNA aptamer. Generally, an inhibitory nucleic acid binds to (e.g., hybridizes with) between about 6 and about 30 (e.g., any integer between 6 and 30, inclusive) contiguous nucleotides of a target RNA (e.g., mRNA). In some embodiments, the inhibitory nucleic acid molecule is an miRNA or an amiRNA, for example an miRNA that targets C9orf72 (the gene encoding pathogenic C9orf72 protein). In some embodiments, the miRNA does not comprise any mismatches with the region of C9orf72 mRNA to which it hybridizes (e.g., the miRNA is “perfected”). In some embodiments, an miRNA comprises between 2 and 20 mismatches (e.g., 2, 3, 4, 5, 6, 7, 8, 9, 10, 11, 12, 13, 14, 15, 16, 17, 18, 19, or 20), such as “bulges”, with the region of C9orf72 mRNA to which it hybridizes. In some embodiments, an miRNA comprises more than 20 mismatches with the region of C9orf72 mRNA to which it hybridizes.

In some embodiments, the inhibitory nucleic acid is an shRNA (e.g., an shRNA targeting C9orf72). In some embodiments, the inhibitory nucleic acid is an miRNA (e.g., an miRNA targeting C9orf72). In some embodiments, expression of one or more inhibitory RNAs of an expression construct is driven by one or more RNA pol III promoters, for example H1 promoter or U6 promoter. Each inhibitory RNA may be driven by a different promoter, or the same promoter.

In some embodiments, an inhibitory nucleic acid is an artificial microRNA (amiRNA). A microRNA (miRNA) typically refers to a small, non-coding RNA found in plants and animals and functions in transcriptional and post-translational regulation of gene expression. MiRNAs are transcribed by RNA polymerase to form a hairpin-loop structure referred to as a pri-miRNAs which are subsequently processed by enzymes (e.g., Drosha, Pasha, spliceosome, etc.) to for a pre-miRNA hairpin structure which is then processed by Dicer to form a miRNA/miRNA* duplex (where * indicates the passenger strand of the miRNA duplex), one strand of which is then incorporated into an RNA-induced silencing complex (RISC). In some embodiments, an inhibitory RNA as described herein is a miRNA targeting C9orf72 (e.g., a dipeptide-repeat region of C9orf72 or a non-dipeptide-repeat region of C9orf72), ATXN2, or TMEM106B.

In some embodiments, an inhibitory nucleic acid targeting C9orf72 comprises a miRNA/miRNA* duplex. In some embodiments, the miRNA strand of a miRNA/miRNA* duplex comprises or consists of the sequence set forth in any one of SEQ ID NO: 24 or 25, 37 or 38, 40 or 41, or a portion thereof. In some embodiments, the miRNA* strand of a miRNA/miRNA* duplex comprises or consists of the sequence set forth in SEQ ID NO: 24 or 25, 37 or 38, 40 or 41 or a portion thereof.

In some embodiments, an inhibitory nucleic acid targeting TMEM106B comprises a miRNA/miRNA* duplex. In some embodiments, the miRNA strand of a miRNA/miRNA* duplex comprises or consists of the sequence set forth in SEQ ID NO: 1 or 7, or a portion thereof. In some embodiments, the miRNA* strand of a miRNA/miRNA* duplex comprises or consists of the sequence set forth in SEQ ID NOs: 1 or 7, or a portion thereof.

In some embodiments, an inhibitory nucleic acid targeting ATXN2 comprises a miRNA/miRNA* duplex. In some embodiments, the miRNA strand of a miRNA/miRNA* duplex comprises or consists of the sequence set forth in any one of SEQ ID NOs: 10-23, or a portion thereof. In some embodiments, the miRNA* strand of a miRNA/miRNA* duplex comprises or consists of the sequence set forth in any one of SEQ ID NOs: 10-23 or a portion thereof.

An artificial microRNA (amiRNA) is derived by modifying native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of an miRNA targeting a gene of interest. An artificial precursor microRNA (pre-amiRNA) is normally processed such that one single stable small RNA is preferentially generated. In some embodiments, scAAV vectors and scAAVs described herein comprise a nucleic acid encoding an amiRNA. In some embodiments, the pri-miRNA scaffold of the amiRNA is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99, pri-MIR-194, pri-MIR-155, and pri-MIR-451. In some embodiments, an amiRNA comprises a nucleic acid sequence targeting C9orf72, ATNX2, or TMEM106B, and an eSIBR amiRNA scaffold, for example as described in Fowler et al. Nucleic Acids Res. 2016 Mar. 18; 44(5): e48.

In some aspects, the disclosure relates to expression constructs comprising combinations of inhibitory RNAs for treatment of neurodegenerative diseases (e.g., ALS/FTD). For example in some embodiments, an expression construct described by the disclosure comprises an inhibitory RNA targeting C9orf72 and an inhibitory RNA targeting Transmembrane Protein 106B (TMEM106B). The order in which the isolated nucleic acid encodes the sequences of the inhibitory nucleic acids can vary. For example, an isolated nucleic acid may, from 5′ end to 3′ end, encode either shRNA targeting C9orf72 and TMEM106B, or TMEM106B and C9orf72.

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculoviral vector, etc.). In some embodiments, the vector is a plasmid (e.g., a plasmid comprising an isolated nucleic acid as described herein). In some embodiments, the vector is a recombinant AAV (rAAV) vector (e.g., an expression construct encoding a transgene flanked by AAV ITRs). In some embodiments, an rAAV vector is single-stranded (e.g., single-stranded DNA). In some embodiments, a vector is a Baculovirus vector (e.g., an Autographa californica nuclear polyhedrosis (AcNPV) vector).

Typically an rAAV vector comprises a transgene flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a ΔITR, for example as described by McCarty et al. (2003) Gene Ther. 10(26): 2112-8.

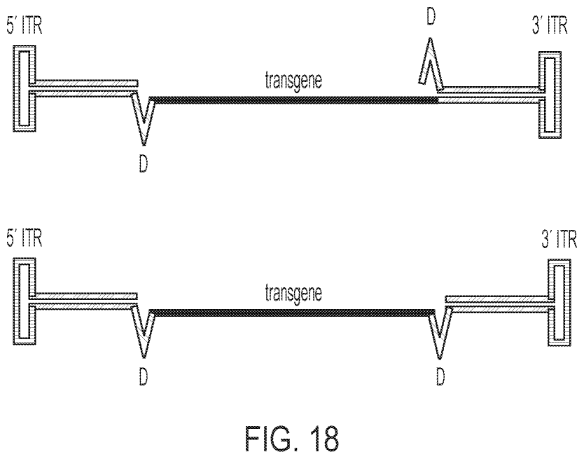

Aspects of the disclosure relate to isolated nucleic acids (e.g., rAAV vectors) comprising an ITR having one or more modifications (e.g., nucleic acid additions, deletions, substitutions, etc.) relative to a wild-type AAV ITR, for example relative to wild-type AAV2 ITR (e.g., SEQ ID NO: 32). The structure of wild-type AAV2 ITR is shown in FIG. 18. Generally, a wild-type ITR comprises a 125 nucleotide region that self-anneals to form a palindromic double-stranded T-shaped, hairpin structure consisting of two cross arms (formed by sequences referred to as B/B′ and C/C′, respectively), a longer stem region (formed by sequences A/A′), and a single-stranded terminal region referred to as the “D” region. (FIG. 18). Generally, the “D” region of an ITR is positioned between the stem region formed by the A/A′ sequences and the insert containing the transgene of the rAAV vector (e.g., positioned on the “inside” of the ITR relative to the terminus of the ITR or proximal to the transgene insert or expression construct of the rAAV vector). In some embodiments, a “D” region comprises the sequence set forth in SEQ ID NO: 30. The “D” region has been observed to play an important role in encapsidation of rAAV vectors by capsid proteins, for example as disclosed by Ling et al. (2015) J Mol Genet Med 9(3).

The disclosure is based, in part, on the surprising discovery that rAAV vectors comprising a “D” region located on the “outside” of the ITR (e.g., proximal to the terminus of the ITR relative to the transgene insert or expression construct) are efficiently encapsidated by AAV capsid proteins than rAAV vectors having ITRs with unmodified (e.g., wild-type) ITRs In some embodiments, rAAV vectors having a modified “D” sequence (e.g., a “D” sequence in the “outside” position) have reduced toxicity relative to rAAV vectors having wild-type ITR sequences.

In some embodiments, a modified “D” sequence comprises at least one nucleotide substitution relative to a wild-type “D” sequence (e.g., SEQ ID NO: 30). A modified “D” sequence may have at least 1, 2, 3, 4, 5, 6, 7, 8, 9, 10, or more than 10 nucleotide substitutions relative to a wild-type “D” sequence (e.g., SEQ ID NO: 30). In some embodiments, a modified “D” sequence comprises at least 10, 11, 12, 13, 14, 15, 16, 17, 18, or 19 nucleic acid substitutions relative to a wild-type “D” sequence (e.g., SEQ ID NO: 30). In some embodiments, a modified “D” sequence is between about 10% and about 99% (e.g., 10%, 15%, 20%, 25%, 30%, 40%, 45%, 50%, 55%, 60%, 65%, 70%, 75%, 80%, 85%, 90%, 95%, or 99%) identical to a wild-type “D” sequence (e.g., SEQ ID NO: 30). In some embodiments, a modified “D” sequence comprises the sequence set forth in SEQ ID NO: 29, also referred to as an “S” sequence as described in Wang et al. (1995) J Mol Biol 250(5): 573-80.

An isolated nucleic acid or rAAV vector as described by the disclosure may further comprise a “TRY” sequence, for example as set forth in SEQ ID NO: 31, as described by Francois, et al. 2005. The Cellular TATA Binding Protein Is Required for Rep-Dependent Replication of a Minimal Adeno-Associated Virus Type 2 p5 Element. J Virol. In some embodiments, a TRY sequence is positioned between an ITR (e.g., a 5′ ITR) and an expression construct (e.g., a transgene-encoding insert) of an isolated nucleic acid or rAAV vector.

In some aspects, the disclosure relates to Baculovirus vectors comprising an isolated nucleic acid or rAAV vector as described by the disclosure. In some embodiments, the Baculovirus vector is an Autographa californica nuclear polyhedrosis (AcNPV) vector, for example as described by Urabe et al. (2002) Hum Gene Ther 13(16): 1935-43 and Smith et al. (2009) Mol Ther 17(11): 1888-1896.

In some aspects, the disclosure provides a host cell comprising an isolated nucleic acid or vector as described herein. A host cell can be a prokaryotic cell or a eukaryotic cell. For example, a host cell can be a mammalian cell, bacterial cell, yeast cell, insect cell, etc. In some embodiments, a host cell is a mammalian cell, for example a HEK293T cell. In some embodiments, a host cell is a bacterial cell, for example an E. coli cell.

rAAVs

In some aspects, the disclosure relates to recombinant AAVs (rAAVs) comprising a transgene that encodes a nucleic acid as described herein (e.g., an rAAV vector as described herein). The term “rAAVs” generally refers to viral particles comprising an rAAV vector encapsidated by one or more AAV capsid proteins. An rAAV described by the disclosure may comprise a capsid protein having a serotype selected from AAV1, AAV2, AAV3, AAV4, AAV5, AAV6, AAV7, AAV8, AAV9, and AAV10. In some embodiments, an rAAV comprises a capsid protein from a non-human host, for example a rhesus AAV capsid protein such as AAVrh.10, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20 25, 50, 100, etc.) amino acid substitutions (e.g., mutations) relative to the wild-type AAV capsid protein from which it is derived.

In some embodiments, rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB). For example, in some embodiments, an rAAV comprises a capsid protein having an AAV9 or AAVrh.10 serotype. In some embodiments, an rAAV comprises an AAV9 variant that crosses the blood-brain barrier, for example AAV-PHP.B serotype, as described by Deverman et al. (2016) Nature Biotechnology 34: 204-209. Generally, production of rAAVs is described, for example, by Samulski et al. (1989) J Viral. 63(9): 3822-8 and Wright (2009) Hum Gene Ther. 20(7): 698-706.

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13(16): 1935-43, Smith et al. (2009) Mol Ther 17(11): 1888-1896, U.S. Pat. Nos. 8,945,918, 9,879,282, and International PCT Publication WO 2017/184879. However, an rAAV can be produced using any suitable method (e.g., using recombinant rep and cap genes).

### Pharmaceutical Compositions

In some aspects, the disclosure provides pharmaceutical compositions comprising an isolated nucleic acid or rAAV as described herein and a pharmaceutically acceptable carrier. As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

Compositions (e.g., pharmaceutical compositions) provided herein can be administered by any route, including enteral (e.g., oral), parenteral, intravenous, intramuscular, intra-arterial, intramedullary, intrathecal, intra-cisterna magna, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

### Methods

The disclosure is based, in part, on compositions for expression of one or more ALS-FTD-associated gene products (or combinations thereof) in a subject that act together (e.g., synergistically) to treat neurodegenerative diseases (e.g., ALS/FTD, etc.). As used herein “treat” or “treating” refers to (a) preventing or delaying onset of neurodegenerative disease (e.g., ALS/FTD, Alzheimer's disease, Gaucher disease, Parkinson's disease, Lewy body dementia, lysosomal storage disease, etc.); (b) reducing severity of neurodegenerative disease; (c) reducing or preventing development of symptoms characteristic of neurodegenerative disease; (d) and/or preventing worsening of symptoms characteristic of neurodegenerative disease. For example, symptoms of ALS/FTD include, for example, motor dysfunction (e.g., paralysis, shaking, rigidity, slowness of movement, difficulty with walking), cognitive dysfunction (e.g., dementia, depression, anxiety), emotional and behavioral dysfunction.

Accordingly, in some aspects, the disclosure provides a method for treating a subject having or suspected of having a neurodegenerative disease, the method comprising administering to the subject a composition (e.g., a composition comprising an isolated nucleic acid or a vector or a rAAV) as described by the disclosure. In some embodiments, the neurodegenerative disease is ALS/FTD, Alzheimer's disease, Gaucher disease, Parkinson's disease, Lewy body dementia, or a lysosomal storage disease.

In some embodiments, a composition is administered directly to the CNS of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, direct injection into the CNS of a subject results in transgene expression (e.g., expression of the first gene product, second gene product, and if applicable, third gene product) in the midbrain, striatum and/or cerebral cortex of the subject.

In some embodiments, compositions as described by the disclosure are administered directly to the cerebrospinal fluid (CSF) of a subject. In some embodiments, direct injection into the CSF results in transgene expression (e.g., expression of the first gene product, second gene product, and if applicable, third gene product) in the spinal cord and/or CSF of the subject. Examples of direct administration to the CSF of a subject include but are not limited to intracisternal injection, intraventricular injection, intralumbar injection, or any combination thereof.

In some embodiments, direct injection to the CNS of a subject comprises convection enhanced delivery (CED). Convection enhanced delivery is a therapeutic strategy that involves surgical exposure of the brain and placement of a small-diameter catheter directly into a target area of the brain, followed by infusion of a therapeutic agent (e.g., a composition or rAAV as described herein) directly to the brain of the subject. CED is described, for example by Debinski et al. (2009) Expert Rev Neurother. 9(10): 1519-27.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV) as described by the disclosure is administered both peripherally and directly to the CNS of a subject. For example, in some embodiments, a subject is administered a composition by intra-arterial injection (e.g., injection into the carotid artery) and by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

The amount of composition (e.g., a composition comprising an isolated nucleic acid or a vector or a rAAV) as described by the disclosure administered to a subject will vary depending on the administration method. For example, in some embodiments, a rAAV as described herein is administered to a subject at a titer between about 10⁹ Genome copies (GC)/kg and about 10¹⁴ GC/kg (e.g., about 10⁹ GC/kg, about 10¹⁰ GC/kg, about 10¹¹ GC/kg, about 10¹² GC/kg, about 10¹² GC/kg, or about 10¹⁴ GC/kg). In some embodiments, a subject is administered a high titer (e.g., >10¹² Genome Copies GC/kg of an rAAV) by injection to the CSF space, or by intraparenchymal injection.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

### EXAMPLES

### Example 1: rAAV Vectors

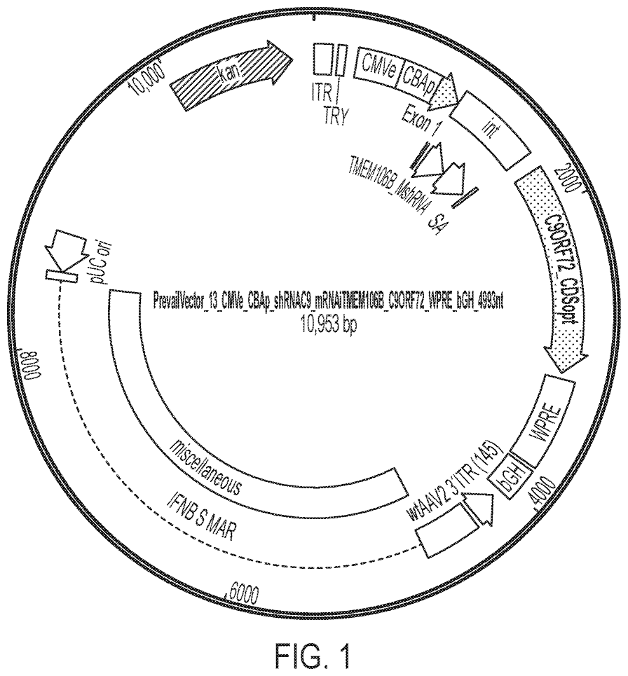

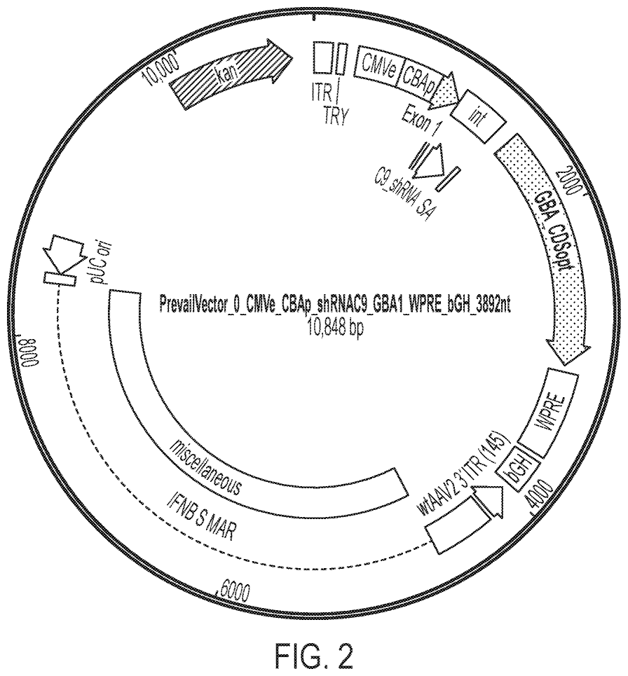

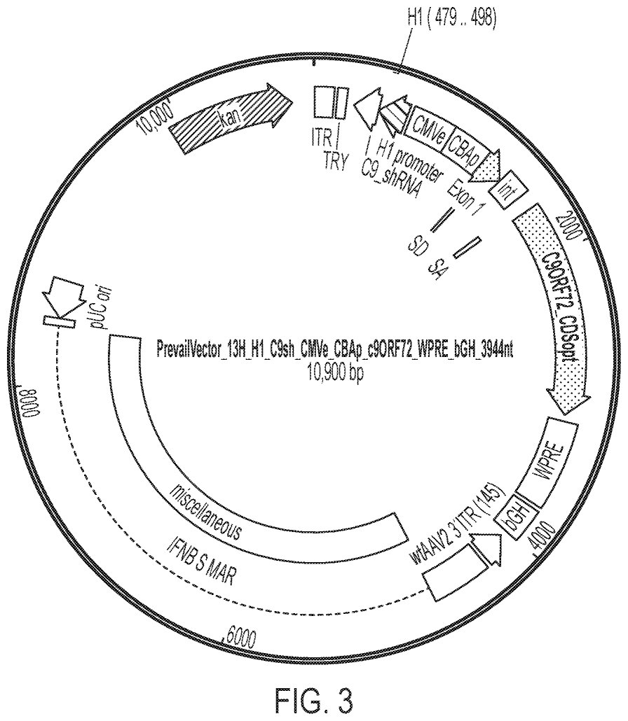

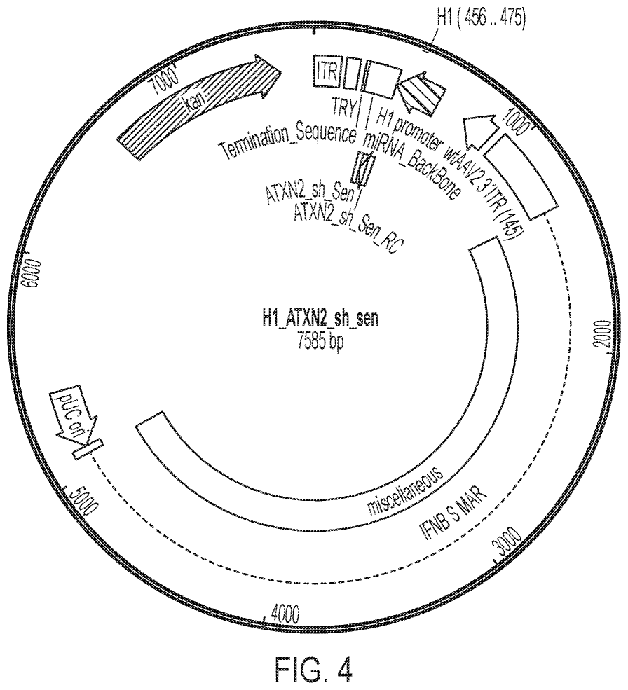

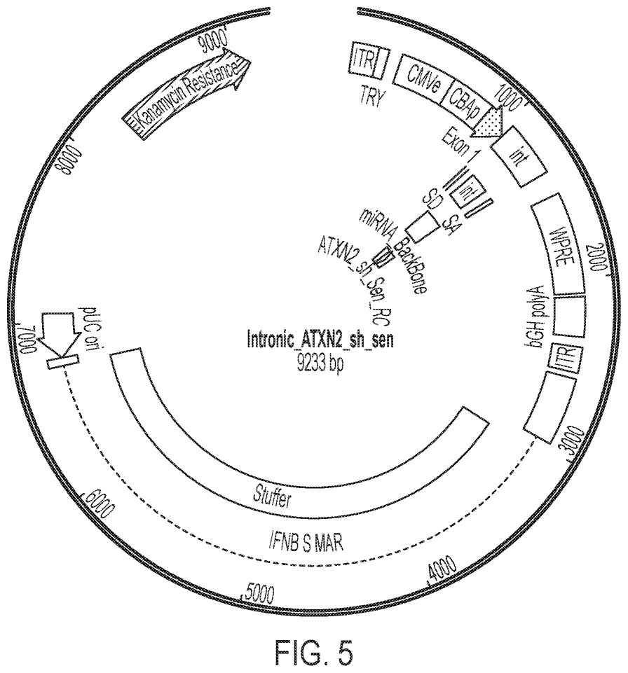

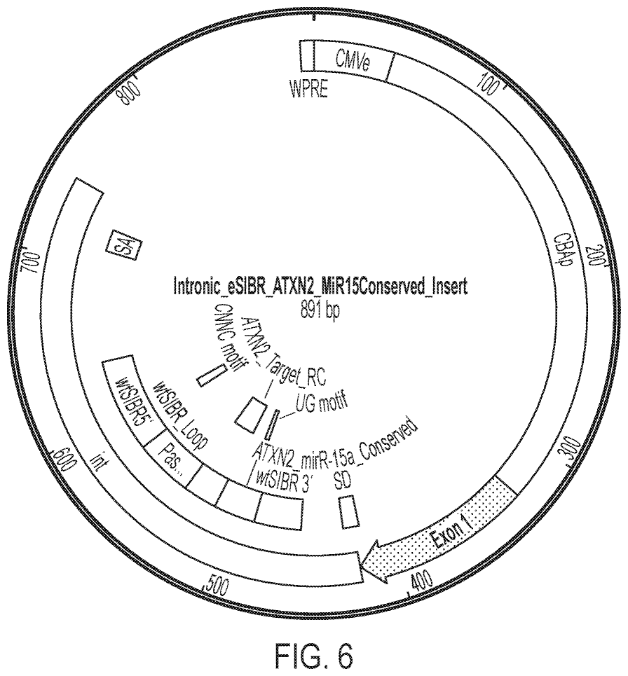

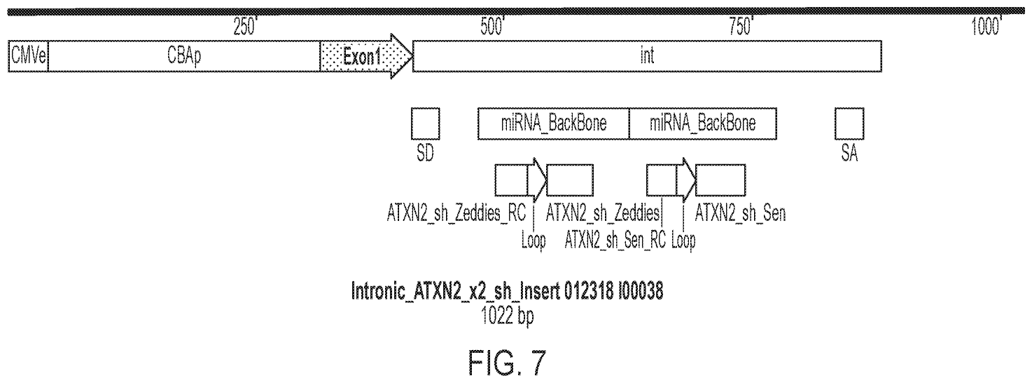

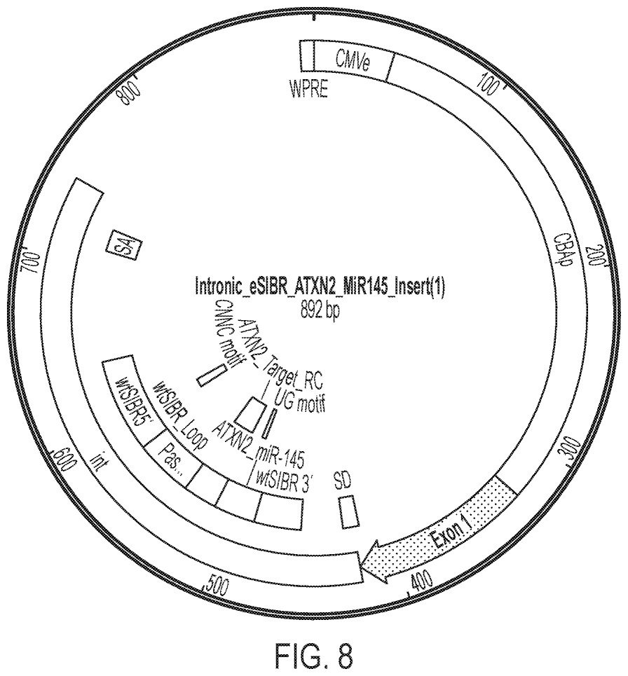

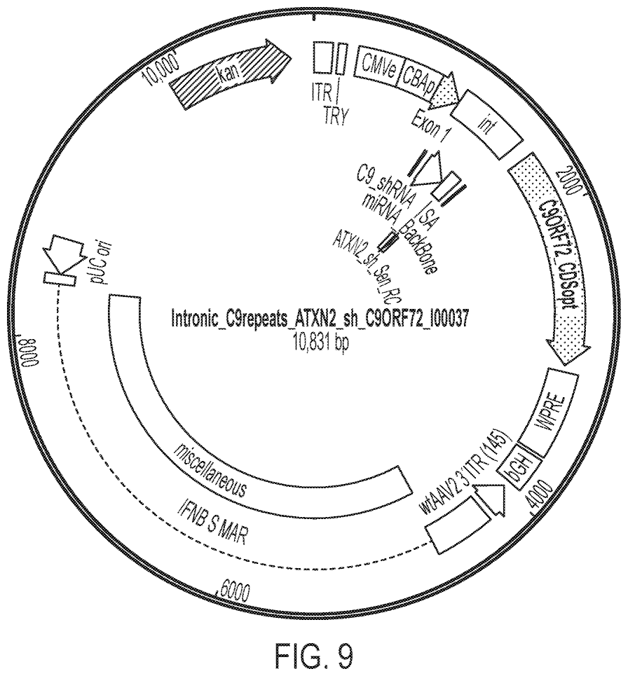

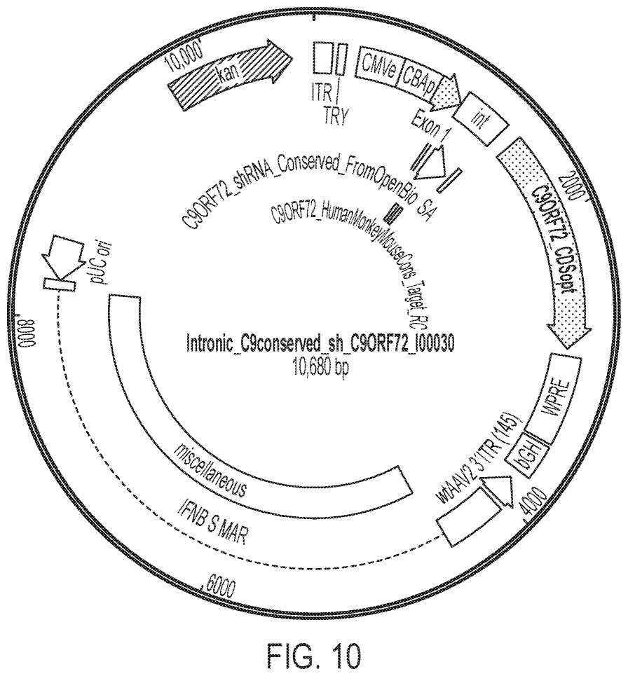

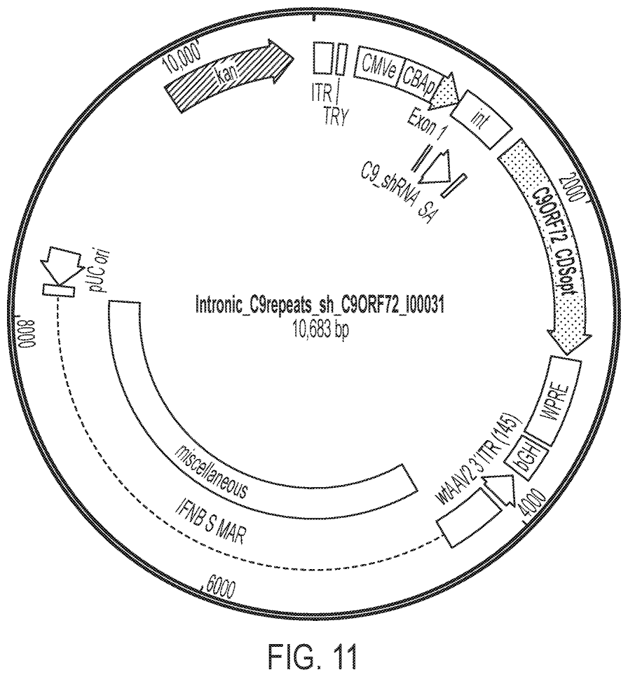

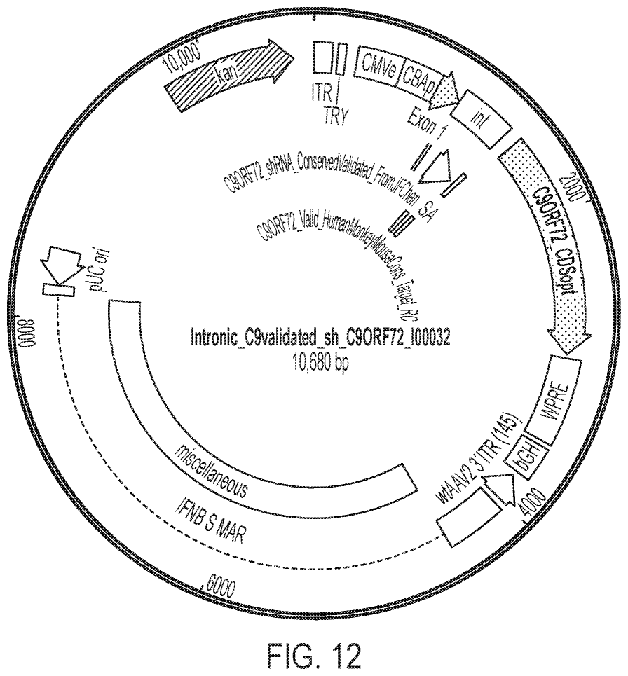

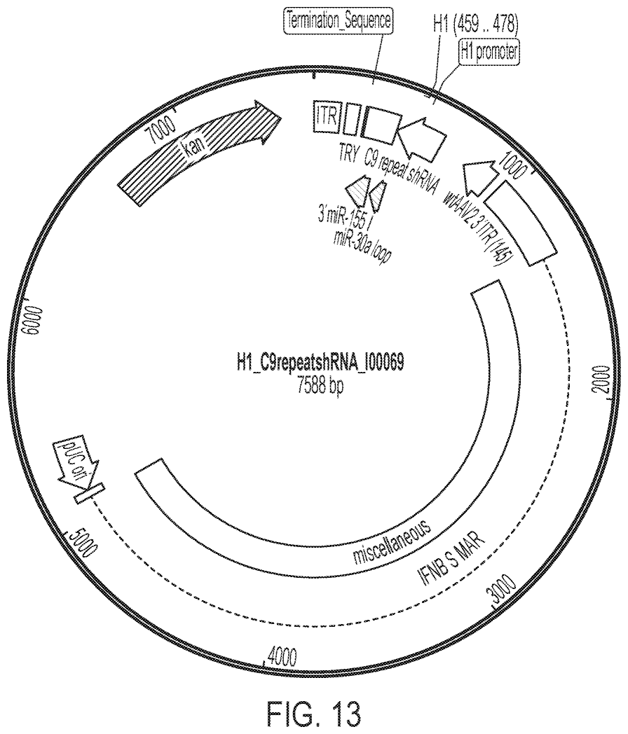

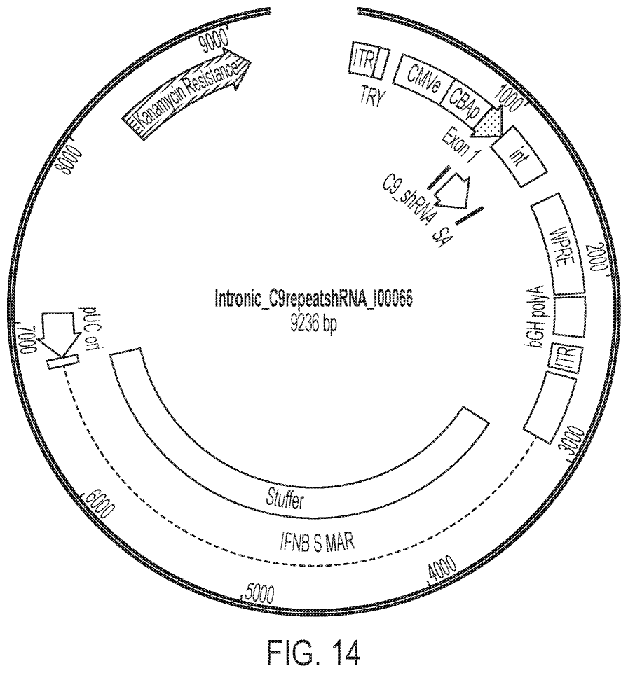

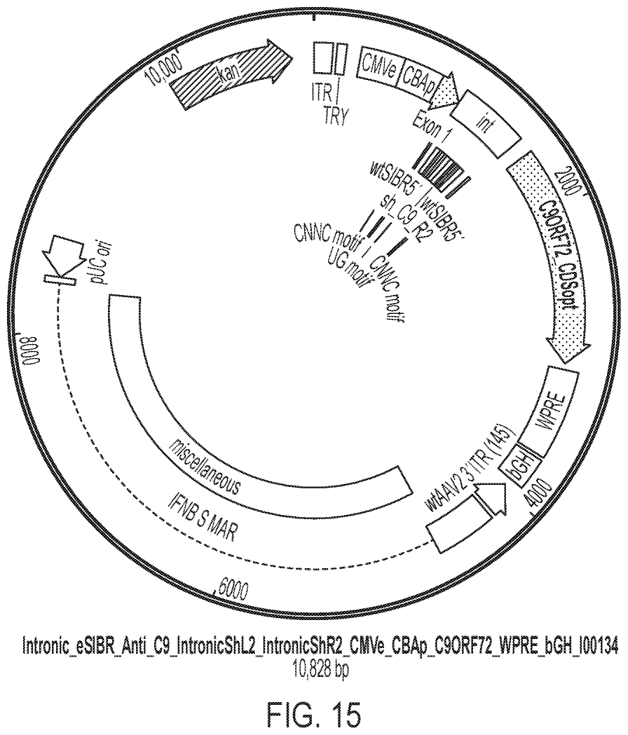

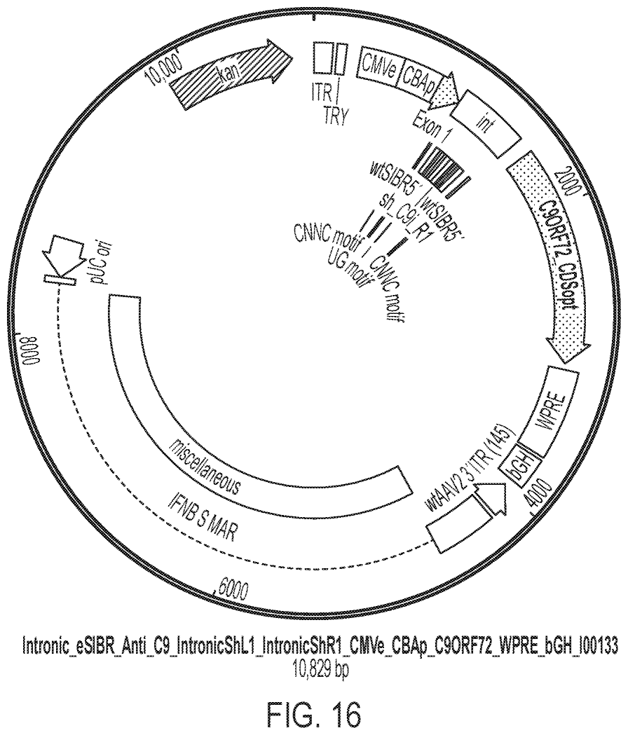

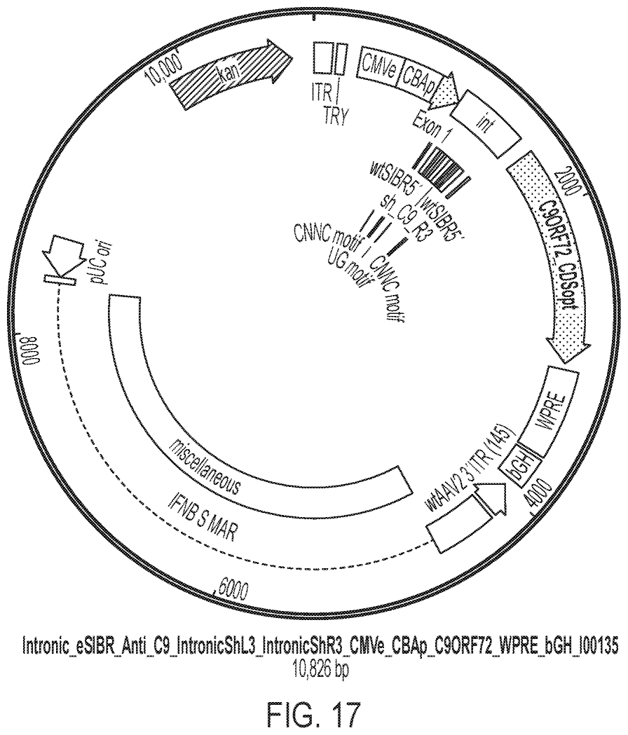

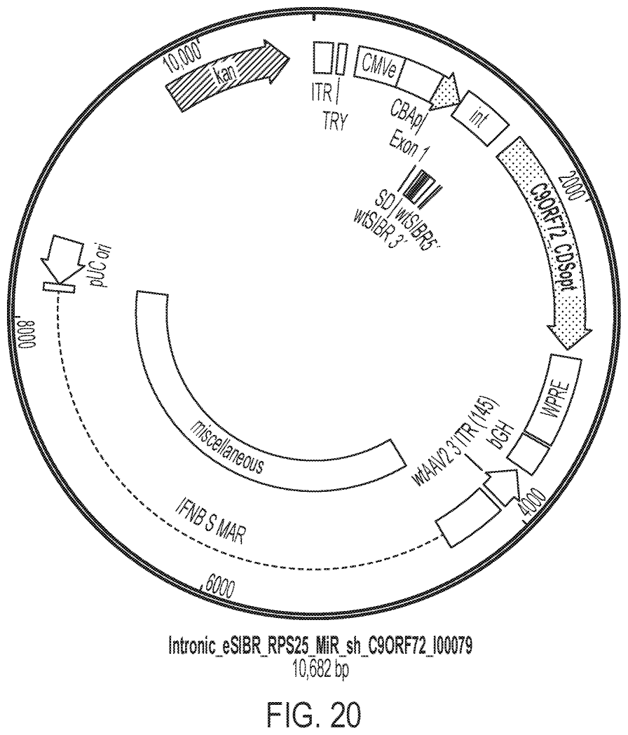

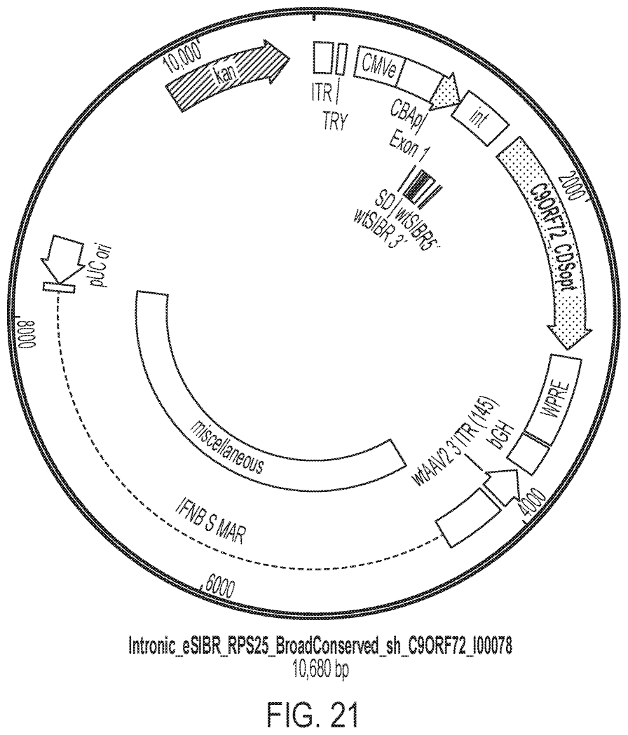

AAV vectors are generated using cells, such as HEK293 cells for triple-plasmid transfection. The ITR sequences flank an expression construct comprising a promoter/enhancer element for each transgene of interest, a 3′ polyA signal, and posttranslational signals such as the WPRE element. Multiple gene products can be expressed simultaneously such as C9orf72 protein or GBA1 protein, and one or more inhibitory nucleic acids (e.g., inhibitory nucleic acids targeting C9orf72 and/or TMEM106B), for example by expression with a single expression cassette or separate expression cassettes. The presence of a short intronic sequence that is efficiently spliced, upstream of the expressed gene, can improve expression levels. Inhibitory RNAs (e.g., miRNAs, shRNAs, etc.) and other regulatory RNAs can potentially be included within these sequences. Examples of expression constructs described by the disclosure are shown in FIGS. 1-17 and 20-21, and in Table 1 below.

TABLE 1

| | | | | | | | | |
|---|---|---|---|---|---|---|---|---|
| | | | | | | | | Length between |
| Name | Promoter 1 | shRNA | CDS1 | PolyA 1 | Promoter 2 | CDS2 | PolyA2 | ITRs |

| | | | | | | |
|---|---|---|---|---|---|---|
| PrevailVector_13_CMVe_{—} | CBA | C9repeats | C9ORF72 | WPRE_b | | 4993 |
| CBAp_shRNAC9_mRNA | | & | | GH | | |
| iTMEM106B_C9ORF72_{—} | | TMEM106B | | | | |
| WPRE_bGH_4993nt | | | | | | |
| PrevailVector_13H_H1_C | Hl | C9repeats | C9orf72 | | CBA | 3944 |
| 9sh_CMVe_CBAp_c9OR | | | | | | |
| F72_WPRE_bGH_3944nt | | | | | | |
| PrevailVector_0_CMVe_{—} | CBA | C9orf72 | GBA1 | WPRE_b | | 3892 |
| CBAp_shRNAC9_GBA1_{—} | | | | GH | | |
| WPRE_bGH_3892nt | | | | | | |
| H1_ATXN2_sh_sen | H1 | ATXN2 | | | | 899 |
| Intronic_ATXN2_sh_sen | CBA | ATXN2 | | WPRE_b | | 2547 |
| | | | | GH | | |
| Intronic_C9repeats_ATN | CBA | C9repeats_A | C9Orf72 | WPRE_b | | 4145 |
| X2_sh_C9ORF71_I00037 | | TXN2 | | GH | | |
| Intronic_eSIBR_Anti_C9_{—} | CBA | C9orf72 | C9orf72 | WPRE_b | | 4140 |
| IntronicShL3_IntronicShR | | | | GH | | |
| 3_CMVe_CBAp_C9ORF | | | | | | |
| 72_WPRE_bGH_I00135 | | | | | | |
| Intronic_eSIBR_Anti_C9_{—} | CBA | C9orf72 | C9orf72 | WPRE_b | | 4143 |
| IntronicShL1_IntronicShR | | | | GH | | |
| 1_CMVe_CBAp_C9ORF | | | | | | |
| 72_WPRE_bGH_I00133 | | | | | | |
| Intronic_C9repeatshRNA_{—} | CBA | C9orf72 | | WPRE_b | | 2250 |
| 100066 | | | | GH | | |
| Intronic_eSIBR_Anti_C9_{—} | CBA | C9orf72 | C9orf72 | WPRE_b | | 4142 |
| IntronicShL2_IntronicShR | | | | GH | | |
| 2_CMVe_CBAp_C9ORF | | | | | | |
| 72_WPRE_bGH_I00134 | | | | | | |
| Intronic_C9repeats_sh_C9 | CBA | C9orf72 | C9orf72 | WPRE_b | | 3997 |
| ORF72_I00031 | | | | GH | | |
| Intronic_C9validated_sh_{—} | CBA | C9orf72 | C9orf72 | WPRE_b | | 3994 |
| C9ORF72_I00032 | | | | GH | | |
| H1_C9repeatshRNA_I00069 | H1 | C9orf72 | | | | 902 |
| Intronic_C9conserved_sh_{—} | CBA | C9orf72 | C9orf72 | WPRE_b | | 3994 |
| C90RF72_I00030 | | | | GH | | |

### Example 2: Cell Based Assays of Viral Transduction into ALS/FTD Cells

Cells characterized by a repeat expansion of C9orf72 are obtained, for example as fibroblasts from ALS/FTD patients, monocytes, or hES cells, or patient-derived induced pluripotent stem cells (iPSCs). These cells accumulate RNA foci and RAN translated proteins.

Using such cell models, cellular pathology is quantified in terms of accumulation of protein aggregates, such as of RAN proteins with an anti-RAN protein antibody, followed by imaging using fluorescent microscopy. Western blotting and/or ELISA is used to quantify abnormal accumulation of RAN proteins.

Therapeutic endpoints (e.g., reduction of ALS/FTD-associated pathology) are measured in the context of expression of transduction of the AAV vectors, to confirm and quantify activity and function. Reduction in endogenous (e.g., pathogenic, repeat expansion-containing) C9orf72 mRNA levels may be quantified, for example, using quantitative RT-PCT (qRT-PCR).

### Example 3: In Vitro Studies

This example describes in vitro testing of C9orf72 rAAV vectors described by the disclosure. Effects of C9orf72 knockdown and overexpression were studied in mammalian cells. Examples of constructs tested are listed in Table 2.

TABLE 2

| | | | | Over- |
|---|---|---|---|---|
| ID | Promoter | Knockdown | Promoter | express |
| I00017 | H1 | C9_sh | CMV | opt-C9 |
| I00018 | CMV | C9_sh, TMEM_mi | CMV | opt-C9 |
| I00030 | CMV_intronic | C9_sh (cons) | CMV | opt-C9 |
| I00031 | CMV_intronic | C9repeat_sh | CMV | opt-C9 |
| I00032 | CMV_intronic | C9_sh(validated) | CMV | opt-C9 |
| I00037 | CMV_intronic | C9r_sh, ATXN_sh | CMV | opt-C9 |

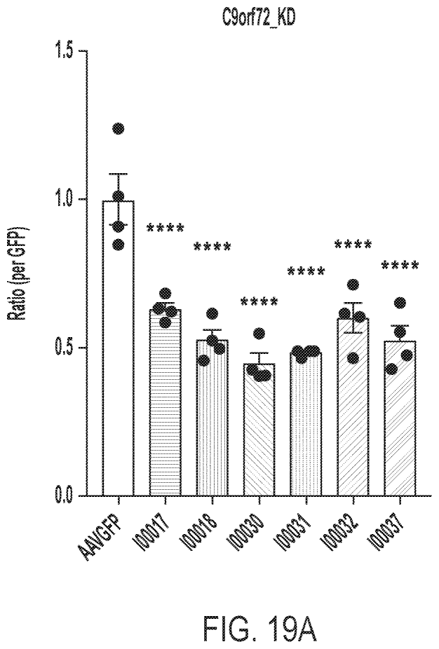

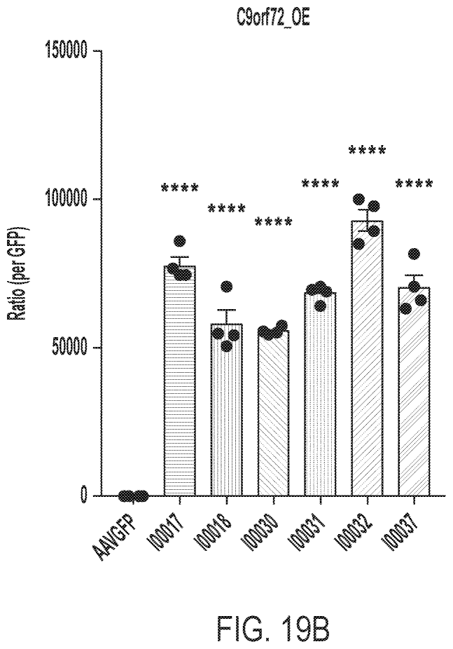

Gene knockdown and overexpression were assayed by quantitative PCR (qPCR) and ELISA. FIG. 19A shows representative data indicating statistically significant silencing of C9orf72 by rAAV vectors. FIG. 19B shows representative data indicating statistically significant increase in wild-type C9orf72 expression after transfection with rAAV vectors.

### SEQUENCES

In some embodiments, an expression cassette encoding one or more gene products (e.g., a first, second and/or third gene product) comprises or consists of (or encodes) a sequence set forth in any one of SEQ ID NOs: 1-62. In some embodiments, a gene product comprises or consists of (or is encoded by) a portion (e.g., fragment) of any one of SEQ ID NOs: 1-62. In some embodiments, the “T” nucleotides in the sequences below are replaced by a “U” nucleotide, for example in the context of an RNA molecule.

The skilled artisan will appreciate that “portions” of the foregoing sequences may be the sequence of the expression cassette (e.g., sequence encoding ITRs, interfering RNAs, coding sequence, regulatory sequence, etc.) that lacks a plasmid backbone (e.g., origin of replication sequence, selection marker sequence, etc.).

1-90. (canceled)

91. An isolated nucleic acid comprising
(i) an expression construct comprises a transgene encoding: (a) a C9Orf72 protein encoded by the sequence set forth in SEQ ID NO: 51 and (b) an inhibitory nucleic acid that inhibits expression or activity of C9orf72, and
(ii) two adeno-associated virus inverted terminal repeats (ITR) sequences flanking the expression construct.

92. The isolated nucleic acid of claim 91, wherein the inhibitory nucleic acid comprises the sequence set forth in SEQ ID NOs: 24, 25, and 37-47.

93. The isolated nucleic acid of claim 91, wherein the transgene is operably linked to a promoter.

94. The isolated nucleic acid of claim 93, wherein the promoter is a chicken beta actin (CBA) promoter.

95. The isolated nucleic acid of claim 93, further comprising a CMV enhancer.

96. The isolated nucleic acid of claim 91, further comprising a Woodchuck Hepatitis Virus Posttranscriptional Regulatory Element (WPRE).

97. The isolated nucleic acid of claim 91, further comprising a Bovine Growth Hormone polyA signal tail.

98. The isolated nucleic acid of claim 91, wherein each ITR sequence is a wild-type AAV2 ITR sequence.

99. The isolated nucleic acid of claim 91, wherein each ITR sequence comprises a “D” region (SEQ ID NO: 30) that is proximal to the transgene.

100. The isolated nucleic acid of claim 91, wherein at least one of the ITR sequences comprises a “D” region (SEQ ID NO: 30) positioned on the outside of the ITR sequence relative to the transgene.

101. The isolated nucleic acid of claim 91, wherein the ITR sequence positioned 5′ relative to the transgene comprises a “D” region (SEQ ID NO: 30) that is proximal to the transgene, and the ITR sequence positioned 3′ relative to the transgene comprises a “D” region (SEQ ID NO: 30) positioned on the outside of the ITR sequence relative to the transgene.

102. The isolated nucleic acid of claim 101, wherein the nucleic acid sequence of the 5′ITR is nucleotides 1-145 of SEQ ID NO: 5 and the nucleic acid sequence of the 3′ ITR is nucleic nucleotides 4070-4214 of SEQ ID NO: 5.

103. The isolated nucleic acid of claim 102, further comprising a TRY region between the 5′ ITR and the transgene, wherein the TRY region has the sequence set forth in SEQ ID NO: 31.

104. A recombinant adeno-associated virus (AAV) vector comprising an expression construct comprises a transgene encoding:
(i) a C9Orf72 protein encoded by the sequence set forth in SEQ ID NO: 51; and
(ii) an inhibitory nucleic acid that inhibits expression or activity of C9orf72;
wherein the expression construct is flanked by two adeno-associated virus (AAV) inverted terminal repeats (ITRs).

105. The rAAV vector of claim 104, wherein the inhibitory nucleic acid comprises the sequence set forth in SEQ ID NOs: 24, 25, and 37-47.

106. The rAAV vector of claim 104 or claim 105, wherein the transgene is operably linked to a promoter.

107. The rAAV vector of claim 106, wherein the promoter is a chicken beta actin (CBA) promoter.

108. The rAAV vector of claim 106, further comprising a CMV enhancer.

109. The rAAV vector of claim 104, further comprising a Woodchuck Hepatitis Virus Posttranscriptional Regulatory Element (WPRE).

110. The rAAV vector of claim 104, further comprising a Bovine Growth Hormone polyA signal tail.

111. The rAAV vector of claim 104, wherein each ITR sequence is a wild-type AAV2 ITR sequence.

112. The rAAV vector of claim 104, wherein each ITR sequence comprises a “D” region (SEQ ID NO: 30) that is proximal to the transgene.

113. The rAAV vector of claim 104, wherein at least one of the ITR sequences comprises a “D” region (SEQ ID NO: 30) positioned on the outside of the ITR sequence relative to the transgene.

114. The rAAV vector of claim 104, wherein the ITR sequence positioned 5′ relative to the transgene comprises a “D” region (SEQ ID NO: 30) that is proximal to the transgene, and the ITR sequence positioned 3′ relative to the transgene comprises a “D” region (SEQ ID NO: 30) positioned on the outside of the ITR sequence relative to the transgene.

115. The rAAV vector of claim 114, wherein the nucleic acid sequence of the 5′ITR is nucleotides 1-145 of SEQ ID NO: 5 and the nucleic acid sequence of the 3′ ITR is nucleic nucleotides 4070-4214 of SEQ ID NO: 5.

116. The rAAV vector of claim 115, further comprising a TRY region between the 5′ ITR and the transgene, wherein the TRY region has the sequence set forth in SEQ ID NO: 31.

117. A recombinant adeno-associated virus (rAAV) comprising:
(i) an AAV capsid protein; and
(ii) the rAAV vector of claim 104.

118. The rAAV of claim 117, wherein the AAV capsid protein is AAV9 capsid protein.

119. A recombinant adeno-associated virus (AAV) vector comprising a nucleic acid comprising, in 5′ to 3′ order:
(a) a 5′ AAV ITR;
(b) a CMV enhancer;
(c) a CBA promoter;
(d) a transgene encoding a C9Orf72 protein, wherein the C9Orf72 protein is encoded by the nucleic acid sequence in SEQ ID NO: 51, and an inhibitory nucleic acid that inhibits expression or activity of C9orf72;
(e) a WPRE;
(f) a Bovine Growth Hormone polyA signal tail; and
(g) a 3′ AAV ITR.

120. The rAAV of claim 119, wherein the inhibitory nucleic acid comprises the sequence set forth in any one of SEQ ID NOs: 24, 25, 37-47

121. A recombinant adeno-associated virus (rAAV) comprising:
(i) an AAV capsid protein; and
(ii) the rAAV vector of claim 119.

122. The rAAV of claim 121, wherein the AAV capsid protein is AAV9 capsid protein.

123. A plasmid comprising the rAAV vector of claim 104.

124. A Baculovirus vector comprising (i) the nucleic acid sequence set forth in SEQ ID NO: 51, and (ii) a nucleic acid sequence encoding an inhibitory nucleic acid that inhibits expression or activity of C9orf72.

125. The Baculovirus vector of claim 124, wherein the inhibitory nucleic acid comprises the sequence set forth in any one of SEQ ID NOs: 24, 25, 37-47.

126. A cell comprising:
(i) a first vector encoding one or more adeno-associated virus rep protein and/or one or more adeno-associated virus cap protein; and
(ii) a second vector comprising an expression construct encoding a C9Orf72 protein encoded by the sequence set forth in SEQ ID NO: 51 and an inhibitory nucleic acid that inhibits expression or activity of C9orf72.

127. The cell of claim 126, wherein the inhibitory nucleic acid comprises the sequence set forth in any one of SEQ ID NOs: 24, 25, 37-47.

128. The cell of claim 126, wherein the first vector is a plasmid and the second vector is a plasmid.

129. The cell of claim 126, wherein the cell is a mammalian cell, optionally wherein the mammalian cell is a HEK293 cell.

130. The cell of claim 126, wherein the first vector is a Baculovirus vector and the second vector is a Baculovirus vector.

131. The cell of claim 130, wherein the cell is an insect cell, optionally wherein the insect cell is a SF9 cell.

132. A method of producing the rAAV of claim 117 or 118, the method comprising:
(i) delivering to a cell a first vector encoding one or more adeno-associated virus rep protein and/or one or more adeno-associated cap protein, and a recombinant AAV vector comprising an expression construct encoding a C9Orf72 protein encoded by the sequence set forth in SEQ ID NO: 51 and an inhibitory nucleic acid that inhibits expression or activity of C9orf72.
(ii) culturing the cells under conditions allowing for packaging the rAAV; and
(iii) harvesting the cultured host cell or culture medium for collection of the rAAV.

133. The method of claim 132, wherein the inhibitory nucleic acid comprises the sequence set forth in any one of SEQ ID NOs: 24, 25, 37-47.

134. A method for treating a subject having or suspected of having a neurodegenerative disease, the method comprising administering to the subject isolated nucleic acid of any one of any one of claims 91 to 103.

135. The method of claim 134, wherein the neurodegenerative disease is amyotrophic lateral sclerosis (ALS) and/or frontotemporal dementia (FTD), or Alzheimer's disease.

136. The method of claim 134, wherein the neurodegenerative disease is ALS and/or FTD.

137. The method of any one of claim 134, wherein the administration comprises direct injection to the CNS of the subject, optionally wherein the direct injection is intracerebral injection, intraparenchymal injection, intrathecal injection, or any combination thereof.

138. The method of claim 134, wherein the direct injection is direct injection to the cerebrospinal fluid (CSF) of the subject, optionally wherein the direct injection is intracisternal injection, intraventricular injection, and/or intralumbar injection.
